(19) United States
(12) Patent Application Publication
(10) Pub. No.: US 2008/0193376 A1
(43) Pub. Date: Aug. 14, 2008

(54) Methods of Enhanced Detection and Therapy of Inflamed Tissues Using Immune Modulation

(75) Inventors: Ahmed Tawakol, Lexington, MA (US); Michael R. Hamblin, Revere, MA (US); Raymond Q. Migrino, Wauwatosa, WI (US); Jeffrey Gelfand, Cambridge, MA (US)

Correspondence Address: EWARDS ANGELL PALMER & DODGE LLP;(Client: MGH, Partners), P.O. BOX 55874, BOSTON, MA, 02205, US

(73) Assignee: The General Hospital Corporation, Boston, MA (US)

(21) Appl. No.: 11/666,572

(22) PCT Filed: Oct. 28, 2004
(86) PCT No.: PCT/US2004/035849

§ 371 Date: Nov. 28, 2007

(51) Int. Cl. A61K 51/02; A61K 49/00; A61K 51/04

(52) U.S. Cl. 424/1.61; 424/9.1; 424/1.73

(57) ABSTRACT

The present invention relates to methods for the detection and therapy of active atheromatous plaques, and in particular vulnerable plaques, whereby immune modulators are used to increase the uptake of diagnostic or therapeutic compositions by the inflammatory cells associated with such plaques.

### RELATED APPLICATIONS/PATENTS & INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Atherosclerosis is a slowly progressing, complex disease that starts during childhood and gradually progresses throughout life. A chief contributor to the pathology of the disease is the formation of “atherosclerotic” or “atheromatous” plaques in luminal walls of arteries (Farb et al. (1995) Circulation 92:1701-1709). These plaques characteristically comprise a fibrous cap surrounding a central core of extracellular lipids and debris located in the central portion of the thickened vessel intima, which is known as the “atheroma.” On the luminal side of the lipid core, the fibrous cap is comprised mainly of connective tissues, typically a dense, fibrous, extracellular matrix made up of collagens, elastins, proteoglycans and other extracellular matrix components. Atheromatous plaques can range from subtle collections of lipid, to large obstructive lesions causing narrowing or “stenosis” of blood vessels. The clinical manifestations of atherosclerosis may be the result of either gradual stenosis of the vessels, which limits blood flow, or the rupture of plaques resulting in the formation of blood clots or “thrombi.”

Any artery in the body can potentially be affected by atherosclerosis. Arguably, the most well known form of atherosclerosis affects the blood vessels supplying the heart, which are the coronary arteries. Atherosclerosis of the coronary arteries is known as coronary artery disease, and is the leading cause of morbidity and mortality in the United States. The narrowing of the coronary arteries as a result of the gradual build up of an atherosclerotic plaque deprives the heart muscles of oxygen and results in angina, often death. However, the major cause of mortality associated with coronary artery disease results not from this gradual narrowing of the blood vessels, but from the sudden rupture of an atherosclerotic plaque. The formation of a thrombus can rapidly block the flow of blood to the heart muscles, causing a heart attack. The rupture of atheromatous plaques located in other arteries can also have serious consequences. For example, the rupture of a plaque in one of the blood vessels supplying the brain can cause a transient ischemic attack or “stroke” and the rupture of a plaque in the peripheral vasculature can block circulation to the limbs or organs, causing serious complications.

Some atheromatous plaques are more likely to rupture than others. Those that are prone to rupture are known as “active” or “vulnerable” plaques, whereas those that are less prone to rupture are known as “stable” plaques. A vulnerable plaque (“VP”) is structurally and functionally distinguishable from a stable atheromatous plaque. A VP is characterized by an abundance of immune cells, such as macrophages and T-lymphocytes, and a thin fibrous cap, whereas stable plaques typically have fewer inflammatory cells and a thickened fibrous cap. The macrophages associated with atheromatous plaques continually phagocytose a form of cholesterol, oxidized low-density lipoprotein (LDL), through cell-surface proteins known as scavenger receptors.

Cells of the immune system are intimately involved in the formation of atheromatous plaques. Two primary classes of inflammatory cells are found in plaques: macrophages and T-lymphocytes. Macrophages are mononuclear phagocytic cells of the immune system. In the presence of soluble cellular factors known as cytokines, precursor cells differentiate into monocytes, which leave the bone marrow and enter the bloodstream, where they further differentiate into mature monocytes. Monocytes circulate in the bloodstream for about 8 h, during which time they enlarge, extravasate into surrounding tissues, and differentiate into macrophages.

Macrophages are morphologically distinct from monocytes such that the cell itself is enlarged while the intracellular organelles become more numerous and complex. The ability of the monocyte to phagocytose increases during differentiation into macrophages, as well as the ability to secrete cytokines. Once the macrophages migrate into tissues, they remain there until stimulated by an antigen. Other cells capable of secreting cytokines, such as helper T-lymphocytes, can also enhance macrophage activity. Macrophages are capable of ingesting exogenous antigens, such as whole microorganisms, insoluble particles (e.g. lipoprotein particles), injured and dead host cells, cellular debris, and activated clotting factors. In the context of atherosclerosis, macrophages can engulf lipid and lipoprotein particles in the atheromatous plaque, causing its differentiation into a specialized type of macrophage known as a foam cell (Li, A. C. et al. 2002 Nat. Med. 8(11): 1235-42).

Some responses of macrophages to atherosclerosis seem to be protective, such as the clearance of oxidized lipoproteins and the efflux of lipoprotein-derived cholesterol to high-density lipoprotein (HDL) acceptors for reverse cholesterol transport. Fatty streaks, which represent the earliest grossly visible atherosclerotic lesions, consist mainly of macrophage foam cells that have taken up massive amounts of cholesterol. The formation of fatty streaks is initiated by the adherence of circulating monocytes to activated vascular endothelial cells at lesion-prone sites within large arteries. Adherent monocytes subsequently migrate into the subendothelial space in response to locally produced chemoattractant molecules, where they further differentiate into macrophages. This program of differentiation includes substantial upregulation of scavenger receptors that normally function in the recognition and internalization of pathogens and apoptotic cells. However, scavenger receptors also recognize altered molecular patterns present on oxidized low-density lipoprotein (oxLDL), and mediate the massive accumulation of cholesterol characteristic of macrophage foam cells.

Fatty streaks can evolve into more complex lesions; this is accelerated by risk factors such as hypertension, hyperlipidemia and hyperglycemia. This evolution is accompanied by the influx of T cells, which secrete cytokines and other regulatory molecules that influence the functional properties of nearby endothelial cells, macrophages and smooth muscle cells. The progression of fatty streaks to more complex lesions involves the migration of smooth muscle cells from the media into the intima, where they accumulate cholesterol and become smooth muscle cell-derived foam cells. The death of lipid-laden foam cells leads to the formation of a necrotic, cholesterol-rich core that becomes walled off by a fibrous cap of extracellular matrix proteins secreted by smooth muscle cells. The lesional macrophages and foam cells also produce a variety of procoagulant substances, including tissue factor, a potent procoagulant. The fibrous cap of an atheromatous plaque is the only barrier separating the powerful coagulants of the lipid core from the circulation. Thus, the thinner the fibrous cap, the greater the propensity for plaque rupture and thrombosis.

Current therapies for atherosclerosis include surgical/interventional techniques and various drug treatments. The surgical/interventional techniques are predominantly designed to ameliorate the occlusive effects of atheromatous plaques on coronary blood flow, i.e. stenosis and include coronary artery bypass surgery and percutaneous transluminal coronary angioplasty. However, these procedures do not always prevent the incidence of acute coronary syndrome. Moreover, at least 50% of patients receiving angioplasty suffer from restenosis and must return for a further procedure between 6 months to one year after the initial procedure.

Post-mortem evidence suggests that vulnerable plaque ruptures occur in areas of the coronary arteries that are less than about 50% stenosed. Thus, angioplasty and bypass procedures, which are carried out on severely stenosed arteries, rarely remove vulnerable plaques or reduce the incidence of acute coronary syndrome (Plutzky (1999) Am J Cardiol 84:15J-20J). Even with currently available therapeutic approaches, such as lipid lowering, angioplasty and bypass, an unacceptably high incidence of acute coronary syndrome remains (Sacks et al. (2000) Circulation 102:1893-1900).

Most current methods of plaque detection, several of which are discussed herein, are inadequate for detecting active atheromatous and vulnerable plaques. Present methods of plaque detection are also inadequate for detecting vulnerable plaques.

Common methods of plaque detection include angiography and angioscopy. Except in rare circumstances, angiography gives almost no information about the characteristics of plaque components. Angiography is only sensitive enough to detect hemodynamically significant lesions (>70% stenosis), which account for only approximately 33% of acute coronary syndrome cases. Angioscopy is a technique based on fiber-optic transmission of visible light that provides a small field of view with relatively low resolution for visualization of interior surfaces of plaque and thrombus. Because angioscopic visualization is limited to the surface of the plaque, it is insufficient for use in detecting actively forming atheromatous and/or vulnerable plaques.

Several methods are being investigated for their ability to identify atheromatous plaques. However, none has proven to be sufficiently sensitive to identify vulnerable plaques or monitor the formation thereof. One such method, intravascular ultrasound (“IVUS”) uses crystals incorporated at catheter tips, providing real-time, cross-sectional and longitudinal, high-resolution images of the arterial wall with three-dimensional reconstruction capabilities. IVUS can detect thin caps and distinguish regions of intermediate density (e.g., intima that is rich in smooth muscle cells and fibrous tissue) from echolucent regions, but current technology does not determine which echolucent regions are composed of cholesterol pools rather than a thrombus, hemorrhage, or some combination thereof. Moreover, the spatial resolution (i.e., approximately 2 cm) does not distinguish the moderately thinned cap from the high risk cap (i.e., approximately 25-75 μm) and large dense calcium deposits produce acoustic echoes which produce “shadows”, so that deeper plaques can not be imaged.

Intravascular thermography is based on the premise that atheromatous plaques with dense macrophage infiltration give off more heat than non-inflamed plaque (Casscells et al. (1996) Lancet. 347:1447-1451). The temperature of the plaque is inversely correlated to cap thickness. However, thermography may not provide information about eroded but non-inflamed lesions, vulnerable or otherwise, having a propensity to rupture.

Optical coherence tomography (“OCT”) measures the intensity of reflected near-infrared light from tissue. It provides images with high resolution that is approximately 10 to 20 times higher than that of IVUS resolution. OCT is primarily used for assessment of atherosclerotic plaque morphology. However, long image acquisition time, high costs, limited penetration and a lack of physiologic data render this approach undesirable for detection of actively forming atheromatous and/or vulnerable plaques.

Raman spectroscopy utilizes the Raman effect, which arises when incident light excites molecules in a sample, subsequently scattering the light. While most of this scattered light is at the same wavelength as the incident light, some is scattered at a different wavelength. This shift in the wavelength of the scattered light is called the Raman shift. The wavelength shift depends on the size, shape, and strength of the molecule. Each molecule has its own distinct “fingerprint”, or Raman shift. Raman spectroscopy is a very sensitive technique and can provide an accurate indication of chemical composition. Conceivably, measurements of lipid:protein ratio might help detect vulnerable plaques by virtue of their high relative lipid content. However, it is unlikely that actively forming and/or vulnerable plaques will be reliably differentiated from stable plaques based solely on such lipid:protein ratios.

The majority of the existing technologies and methods used to date are based on detecting structural elements of plaques and/or structural changes in the blood vessels, such as stenosis. This means that such methods may not be useful in detecting small, but actively forming or vulnerable plaques. Furthermore, the majority of vascular detection methods known in the art involve the use of external imaging devices, such as gamma or positron cameras. The usefulness of such methods in detecting small and/or vulnerable plaques is limited by difficulties in distinguishing such plaques from the background of the surrounding tissue. Although 3D imaging using positron emission tomography (PET) and Single-Photon Computed Tomography (SPECT) is presently in use, the small size of the arteries as compared to the scatter from the large surrounding tissues lowers the utility of these imaging modalities as well.

Photodynamic therapy (“PDT”) employs photoactivatable compounds known as photosensitizers to selectively target and destroy cells. Therapy involves delivering visible light of the appropriate wavelength to excite the photosensitizer molecule to the excited singlet state. This excited state can then undergo intersystem crossing to the slightly lower energy triplet state, which can then react further by one or both of two pathways, known as Type I and Type II photoprocesses (Ochsner (1997) J Photochem Photobiol B 39:1-18). The Type I pathway involves electron transfer reactions from the photosensitizer triplet to produce radical ions that can then react with oxygen to produce cytotoxic species such as superoxide, hydroxyl and lipid derived radicals. The Type II pathway involves energy transfer from the photosensitizer triplet to ground state molecular oxygen (triplet) to produce the excited state singlet oxygen, which can then oxidize many biological molecules such as proteins, nucleic acids and lipids, and lead to cytotoxicity.

Photodynamic therapy has been applied in cardiovascular medicine for two broad indications: treatment of atherosclerosis (“photoangioplasty”) and inhibition of restenosis due to intimal hyperplasia after vascular interventions (Rockson et al. (2000) Circulation 102:591-596, U.S. Pat. Nos. 5,116,864, 5,298,018, 5,308,861, 5,422,362, 5,834,503 and 6,054,449). Hematoporphyrin derivative (“HpD”) was the first of a number of photosensitizers with demonstrable, selective accumulation within atheromatous plaques (Litvack et al. (1985) Am J Cardiol 56:667-671). Subsequent studies have underscored the affinity of porphyrin derivatives for atheromatous plaques in rabbits and miniswine. There is maximal photosensitizer accumulation within the arterial intimal surface layers, which is diminished in comparison to the arterial media. Both HpD and Photofrin, a more purified derivative of HpD, also display in vitro preferential uptake by human atheromatous plaques. However, there is generally a relative lack of selectivity of most photosensitizers for atheromatous plaques and more particularly for vulnerable plaques.

Thus, there are deficiencies associated with current methods available for the detection and treatment of atheromatous and vulnerable plaques. In particular, current diagnostic methods are not able to reliably detect those plaques that are most prone to rupture, and are therefore the most life-threatening. There exists a need in the art for improved methods of detection and treatment of rupture-prone atheromatous plaques, allowing for localized stabilization and reducing the risk of thrombus formation.

### SUMMARY OF THE INVENTION

It has now been shown that modulators of the immune system can increase the selective targeting of diagnostic and therapeutic compositions to active and vulnerable atheromatous plaques. Methods of the present invention employ immune modulators to increase the uptake of diagnostic or therapeutic compositions by inflammatory cells associated with active atheromatous and vulnerable plaques, thereby improving diagnosis and therapy.

In one aspect of the present invention, a method for identifying active atheromatous and vulnerable plaque in a subject is provided, the method comprising the steps of:
a) administering a diagnostic composition;
b) administering an immune modulator that increases localization of the diagnostic composition to inflammatory cells of the plaque;
c) detecting a sufficient amount of the diagnostic composition to thereby identify the plaque in the subject.

The diagnostic composition can be comprised of a diagnostic agent coupled to a molecular carrier. In some embodiments, the diagnostic agent is internalized by the inflammatory cells.

Diagnostic agents can be but are not limited to photosensitizers, radiolabeled markers (e.g., radionuclides, paramagnetic contrast agents, β-emitters) and fluorescent markers.

Diagnostic methods of the present invention can comprise further steps, wherein a vulnerable plaque is identified, and distinguished from other lesions within the blood vessel, including stable atheromatous plaques. Accordingly, in another aspect of the present invention, a method for identifying active atheromatous and vulnerable plaque in a blood vessel is provided, the method comprising the steps of:
a) administering a diagnostic composition;
b) administering an immune modulator that increases localization of the diagnostic composition to inflammatory cells of the plaque;
c) comparing a signal emitted by the diagnostic composition in one area of the blood vessel to a signal emitted by the diagnostic composition in another area of the blood vessel; and
d) determining the location of the greater amount of signal to thereby identify the plaque in the subject.

In yet another aspect of the present invention, a method for treating active atheromatous and vulnerable plaque in a subject in need thereof is provided, the method comprising the steps of:
a) administering a therapeutic composition;
b) administering an immune modulator that increases localization of the therapeutic composition to inflammatory cells of the plaque; and
c) stabilizing, reducing or eliminating the plaque, thereby treating the subject for active atheromatous and vulnerable plaque.

The therapeutic composition can be comprised of a therapeutic agent coupled to a molecular carrier. In some embodiments, the therapeutic agent is internalized by the inflammatory cells.

Molecular carriers can be but are not limited to serum proteins, receptor ligands, microspheres, liposomes, antibodies, growth factors, peptides, hormones and lipoproteins. In specific embodiments, the molecular carriers are targeted to scavenger receptors, T-cells, macrophages, foam cells, or lipid pools of the atheroma.

The immune modulator can be but is not limited to a colony stimulating factor, interleukin, interferon, chemokines, chemoattractant, growth factor, inhibitory factor, bacterially derived epitope or signal transduction molecule.

In specific embodiments, the immune modulator is GM-CSF, M-CSF, G-CSF, interleukins-1 through 29 (abbreviated IL-1, IL-2, and so on), TNFα, formyl-methionine-leucine-phenylalanine (fMLP), endotoxins, and lipopolysaccharide (LPS), phorbol 12-myristate-13-acetate, interferon α, interferon β, interferon γ, CD40, ligands of CD40 (e.g., gp39), MCP-1 through 5, bFGF, muramyl dipeptide, urokinase, a C-, CC-, CXC- or CX3C family member, RANTES, GRO α, β, γ, I-TAC, MIG-1, LIF, oncostatin M, TGF β, TIMP, MCF, and MIP-1 through 5, or α, β, δ, γ isoforms thereof.

The inflammatory cells in which diagnostic and therapeutic compositions of the invention are localized, and optionally internalized, include but are not limited to smooth muscle cells, dendritic cells, follicular dendritic cells, Langerhans cells, interstitial, interdigitating, blood, and veiled dendritic cells, leukocytes, natural killer cells, lymphocytes, monocytes, macrophages, alveolar macrophages, microglia, mesangial cells, histiocytes, Kupffer cells, foam cells, mast cells, endothelial cells, megakaryocytes, platelets, erythrocytes and polymorphonuclear cells.

Diseases and conditions for which subjects of the present invention may undergo treatment or diagnosis include but are not limited to coronary artery disease, atherosclerosis, stenosis and thrombus formation.

In yet another aspect of the present invention, a method for identifying and stabilizing active atheromatous and vulnerable plaque in a subject with the use of photodynamic means is provided, the method comprising the steps of:
a) administering at least one photosensitizer;
b) administering an immune modulator that increases localization of the photosensitizer to inflammatory cells of the plaque;
c) detecting a sufficient amount of the photosensitizer to thereby identify the plaque; and
d) irradiating the photosensitizer to produce a phototoxic species that stabilizes the plaque in the subject.

In yet another aspect, the present invention provides a kit for identifying inflamed tissue comprising a diagnostic agent, an immune modulator and instructions for using the diagnostic agent and the immune modulator to identify inflamed tissue in accordance with the methods of the invention.

In one embodiment, the kit of includes a detector for detecting the diagnostic agent.

In yet another aspect, the present invention provides a kit for treating active atheromatous and vulnerable plaque in a subject in need thereof comprising a therapeutic agent, an immune modulator and instructions for treating active atheromatous and vulnerable plaque using the therapeutic agent in the subject in accordance with the methods of the invention.

In yet another aspect, the present invention provides a kit for detecting and treating active atheromatous and vulnerable plaque in a subject in need thereof comprising a photosensitizer, an immune modulator, and instructions for using the photosensitizer and the immune modulator to detect and treat active atheromatous and vulnerable plaque in a subject in accordance with the methods of the invention.

Other aspects of the invention are described in or are obvious from the following disclosure, and are within the ambit of the invention.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 provides a comparison of a vulnerable plaque and a stable plaque. Vulnerable plaques feature a thin, fibrous cap, as well as the presence of immune cells such as T-lymphocytes, macrophages, and foam cells.

FIG. 2 illustrates cross sections of rabbit aortas that are either healthy or atherosclerotic. The top panels show Masson's Trichrome Staining of rabbit aortas, while the lower panels illustrate elastic tissue staining.

FIG. 3 shows the activity of fluorodeoxyglucose (FDG) compared to standard well counting, as detected by an intravascular β-catheter.

FIG. 4 shows FDG detected by the intravascular β-catheter in regions containing atherosclerotic plaques compared to non-plaque regions.

FIG. 5 shows the accumulation of a radiolabeled chemotactic peptide, CPRA, within excised atherosclerotic and control rabbit aortas.

FIG. 6 shows the scheme for preparing chlorinₑ₆ photosensitizer conjugates.

FIG. 7 shows BSA-cₑ₆ purified from unreacted cₑ₆-NHS ester using a Sephadex G50 column and acetone precipitation (7A: Thin Layer Chromatography; 7B: SDS-PAGE gel visualized by fluorescence (left) and Coomassie stain (right) before acetone precipitation; 7C: SDS-PAGE gel visualized by fluorescence (left) and Coomassie stain (right) after acetone precipitation).

FIG. 8 shows the UV-visible absorption spectra of the purified mal-BSA-cₑ₆ conjugates and free cₑ₆.

FIG. 9 shows an optical multichannel analyzer used for fluorescence localization within ex vivo aortas.

FIG. 10 shows an analysis of aortic sections from rabbits injected with or without conjugates about 24 hours after injection of the conjugate (Row 1: confocal fluorescence, red signal indicates chlorinₑ₆, green signal indicates elastic lamina auto-fluorescence; Row 2: fluorescence emission spectra of intimal surface of aortic segments ex vivo; Row 3: Hematoxylin and eosin staining of formalin fixed paraffin embedded aortic segments; Row 4: Verhoeff's elastic tissue stain). Column 1 shows an atherosclerotic rabbit with no injection of conjugate. Column 2 shows a normal non-atherosclerotic rabbit injected with conjugate. Column 3 shows an atherosclerotic rabbit injected with conjugate.

FIG. 11 shows the contrast between a large aortic plaque and an area of the abdominal aorta 5 mm beneath the plaque (Panel 1), between the balloon injured iliac artery and the contralateral normal artery in the same rabbit (Panel 2), and between the plaque-laden aorta of an atherosclerotic rabbit and the same area of the aorta in a normal rabbit (Panel 3).

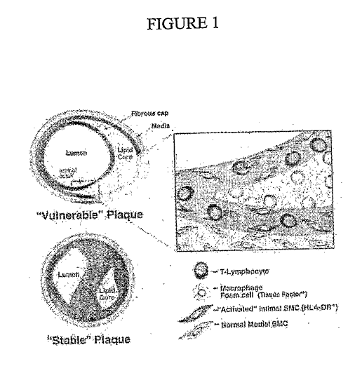

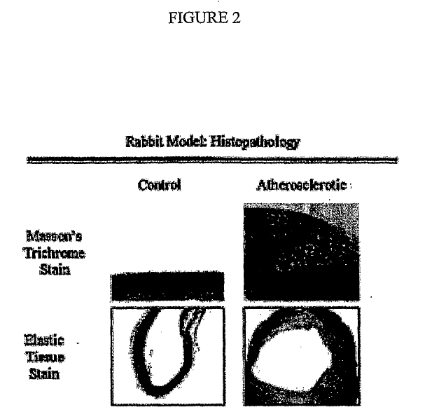

### DETAILED DESCRIPTION OF THE INVENTION

### I. Definitions

Atherosclerosis is caused by the formation of “atherosclerotic-” or “atheromatous-plaques” in the luminal walls of blood vessels. Atherosclerosis often affects the coronary arteries, causing coronary artery disease, but it can also affect blood vessels elsewhere in the body.

Atherosclerotic diseases are the result of either the occlusion or “stenosis” of blood vessels, or the rupture of atheromatous plaques leading to thrombus formation. “Stenosis” refers to a constriction or decrease in blood vessel diameter, which in the case of atherosclerosis is typically caused by the presence of a large atheromatous plaque in the vessel wall that forms a physical barrier restricting the flow of blood. As used herein, the term, “thrombus” refers to a clot of blood formed within a blood vessel. In the case of atherosclerosis, thrombi are caused by the release of powerful coagulants into the bloodstream by the rupture of an atheromatous plaque.

In the context of this application, an “inactive or stable atheromatous plaque” comprises a thick fibrous cap, preferably greater than about 200 microns thick, a small lipid pool or the absence thereof, which only slowly accumulates lipids, if at all, and causes less than about 50% stenosis. Preferably, less than about 50% stenosis is maintained for one month, more preferably less than about 50% stenosis is maintained for six months and even more preferably less than about 50% stenosis is maintained for one year.

Vulnerable plaques, or VPs, comprise an abundance of inflammatory cells, a large lipid pool, and a thin fibrous cap. A vulnerable plaque comprises a fibrous cap that is less than about 150 microns thick. Preferably, a vulnerable plaque comprises a fibrous cap that is less than about 100 microns thick (e.g., between about 60 to about 100 microns thick). A vulnerable plaque comprises a macrophage and/or monocyte content that is greater than about 10%. Preferably, a vulnerable plaque comprises a macrophage and/or monocyte content that is greater than about 25%. A vulnerable plaque comprises a lipid content that is greater than about 10%. Preferably, a vulnerable plaque comprises a lipid content that is greater than about 25%.

An “active atheromatous plaque” comprises a plaque accumulating aggregated platelets and monocytes, such that greater than about 50% stenosis is achieved within about one month of the onset of growth. Preferably about 50% stenosis is achieved within six months of the onset of growth, and more preferably about 50% stenosis is achieved within one year of the onset of growth. The onset of active atheromatous plaque growth can follow a surgical procedure, such as angioplasty.

Active atheromatous and vulnerable plaques are collectively referred to herein as “unstable plaques.”

“Inflammatory cells” are cells contributing to growth of an active atheromatous or vulnerable plaque that can include but are not limited to smooth muscle cells, dendritic cells, follicular dendritic cells, Langerhans cells, interstitial, interdigitating, blood, and veiled dendritic cells, leukocytes, natural killer cells, lymphocytes (B-lymphocytes and T-lymphocytes), monocytes, macrophages, foam cells, tissue-specific macrophages such as alveolar macrophages, microglia, mesangial cells, histiocytes, and Kupffer cells, mast cells, endothelial cells, megakaryocytes, platelets, erythrocytes and polymorphonuclear cells (e.g., granulocytes such as basophils, eosinophils, neutrophils).

As used herein, the term “inflammatory components” includes inflammatory cells, lipids, procoagulants (e.g., tissue factor) and enzymes or other agents that promote inhibition of extracellular matrix production or degradation of extracellular matrix components (e.g., proteases).

The term “immune modulator” refers to any molecule capable of activating an inflammatory cell. “Activation” of inflammatory cells is a phenomenon well known in the art, involving an increase in metabolic and signaling activity by inflammatory cells in response to a stimulus. One manifestation of activation is an increase in ligand uptake and receptor turnover. Other manifestations include changes in cell size, mobility, complexity and proliferative capacity, as well as permanent or transient changes in gene expression. Activated inflammatory cells have increased cell surface binding and internalization capacity. Thus, by way of activation, immune modulators can “localize” such diagnostic and therapeutic agents of the invention to inflammatory cells of active atheromatous and vulnerable plaques.

A “molecular carrier” refers to a biomolecule with targeting specificity for active atheromatous or vulnerable plaques. Molecular carriers are delivery vehicles that “target” therapeutic or diagnostic agents of the invention to inflammatory cells or other inflammatory components for which they have affinity.

As used herein, a “β-emitter” is a composition, such as a radionuclide or a paramagnetic contrast agent, that emits electron or positron rays (“β rays”).

The term “photosensitizer” refers to a photoactivatable compound, or a biological precursor thereof, that produces a reactive species (e.g., oxygen) having a photochemical (e.g., cross linking) or phototoxic effect on a cell, cellular component or biomolecule.

The term “obtaining” as in “obtaining the diagnostic agent” is intended to include purchasing, synthesizing or otherwise acquiring the diagnostic agent (or indicated substance or material).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### II. Methods and Compositions of the Invention

The present invention provides methods for detecting and/or treating active atheromatous and vulnerable plaque by co-administering a diagnostic and/or therapeutic composition with an immune modulator, whereby the immune modulator stimulates the localization, and optionally internalization, of the composition by inflammatory cells associated with the plaque. In this way, methods of the present invention enable the enhanced uptake of diagnostic and/or therapeutic compositions by active atheromatous and vulnerable plaques, thus enhancing their detection and treatment. Use of the immune modulators to stimulate uptake minimizes the effect of non-specific uptake by stable plaque regions of the blood vessel. This decrease in the “signal-to-noise” ratio increases the specificity of rupture prone plaque detection and treatment.

A therapeutic composition according to the invention can contain a suitable concentration of an active agent (referred to herein as a therapeutic agent) and may also comprise certain other components. For example, in some embodiments, therapeutic compositions of the present invention are formulated with pharmaceutically acceptable carriers or excipients, such as water, saline, aqueous dextrose, glycerol, or ethanol, and may also contain auxiliary substances such as wetting or emulsifying agents, and pH buffering agents in addition to the therapeutic agent. The therapeutic composition can also be comprised of a therapeutic agent coupled to a molecular carrier that has an affinity for inflammatory cells or inflammatory components.

Therapeutic agents can be any treatment modality known in the art for the treatment of active atheromatous plaques and/or vulnerable plaques, including but not limited to statins (e.g., atorvastatin, or pravastatin), cholesterol lowering drugs, 3-hydroxy-methyl-glutaryl coenzyme A (HMG-CoA) reductase inhibitors, nicotinic acid, bile acid-binding resins such as cholestyramine and colestipol, aspirin, anti-inflammatory agents, bisphosphonates, eicosapentaenoic acid, docosahexaenoic acid, angiotensin converting enzyme (ACE) inhibitors (e.g., ramipril), biomolecules (e.g., thrombin-activatable fibrinolysis inhibitor, Angpt13, or Apo-A1 mimetic peptide,), platelet IIb and IIIa inhibitors such as eptifibatide, tirofiban, clopedigrol, and abciximab, photosensitizers, clot-reducing agents (e.g., tissue plasminogen activator), or those described in WO 01/04819 and U.S. Pat. No. 6,183,752, the contents of which are incorporated herein by reference.

The present invention further provides methods to identify active atheromatous and vulnerable plaques by targeting diagnostic compositions to inflammatory cells of the plaques and employing one or more additional means to identify the diagnostic composition. Like therapeutic compositions of the invention, diagnostic compositions can comprise a suitable concentration of a diagnostic agent and can also comprise certain other components such as pharmaceutically acceptable carriers or excipients, such as water, saline, aqueous dextrose, glycerol, or ethanol, and may also contain auxiliary substances such as wetting or emulsifying agents, and pH buffering agents. The diagnostic composition can also be comprised of a diagnostic agent coupled to a molecular carrier. Diagnostic compositions of the invention emit signals that can be detected by standard means known in the art, including but not limited to thermal detection, intravascular ultrasound, intravascular thermography, Raman spectroscopy, angioscopy, near-infrared spectroscopy, intravascular nuclear probes, intravascular electrical impedance imaging, elastography, optical coherence tomography, magnetic resonance imaging, positron emission tomography, single photon emission computed tomography, or other detection modalities known in the art.

Methods of the present invention can be used to improve efficacy of surgical and drug treatments that target atheromatous plaques. For example, diagnostic and/or therapeutic methods of the invention can be carried out together with treatment schemes known in the art. Several such treatment schemes are contemplated, including percutaneous transluminal coronary angioplasty (“balloon” angioplasty), excimer laser angioplasty such as thermal, photothermal, and photoablative laser angioplasty, atherectomy, percutaneous transmyocardial revascularization, and rotablation. Additionally, stents, drug-coated stents, and drug-eluting stents can also be used in combination with the instant invention or with the treatment and diagnostic schemes described herein. Typical drugs that can coat or be eluted from the stents include the drugs well known in the art for the treatment of atherosclerosis, but also include immunosuppressive agents such as sirolimus, everolimus, actinomycin D, and paclitaxel.

A. Immune Modulators

Immune modulators of the present invention encompass diverse categories and sub-categories of molecules known in the art to activate inflammatory cells, including the colony stimulating factors, the interleukins, the interferons, the chemokines/chemoattractants, growth factors, inhibitory factors, peptides and bacterially derived epitopes, and signal transduction molecules. Immune modulators can be soluble or membrane-bound and can consist, for example, not only of receptors, but also of the ligands for receptors.

The colony stimulating factors include but are not limited to granulocyte-macrophage colony stimulating factor (GM-CSF), macrophage colony stimulating factor (M-CSF) and granulocyte colony stimulating factor (G-CSF).

The interleukins include but are not limited to interleukins-1 through 29 (abbreviated IL-1, IL-2, and so on).

The interferons include but are not limited to interferon α, interferon β, interferon γ, isoforms and splice variants thereof.

The chemokines and chemoattractants include but are not limited to categories and subcategories of the C-, CC-, CXC-, and CX3C family members, RANTES (regulated upon activation normal T-cell expressed and presumably secreted), interferon-inducible T-cell alpha chemoattractant (1-TAC), monocyte chemoattractant protein-1 through 5 (MCP-1 through 5) and macrophage chemotactic factor (MCF).

The growth factors include but are not limited to growth regulated oncogenes (GRO) α, β, γ, basic fibroblast growth factor (bFGF) and transforming growth factor β (TGF β).

The inhibitory factors include but are not limited to tissue inhibitors of metalloproteinases (TIMP), leukemia inhibitory factor (LIF), Membrane inhibitor of reactive lysis (MIRL), anaphylatoxin inactivator, C1 inhibitor (C1Inh) and oncostatin M.

The peptides and bacterially derived epitopes include but are not limited to formyl-methionine-leucine-phenylalanine (fMLP), endotoxins, muramyl dipeptide and lipopolysaccharide (LPS).

The signal transduction molecules include but are not limited to tumor necrosis factor α (TNFα), phorbol esters such as phorbol 12-myristate-13-acetate (PMA), CD40, ligands of CD40 such as gp39, urokinase, prolactin (PRL), monokine induced by gamma-interferon (MIG-1), macrophage inflammatory protein-1 through 5 including isoforms α, β, δ, γ (MIP-1 through 5), opsonins, complement proteins C1 through C9 as well as any products of proteolysis, regulators of complement proteins such as Factor B, Factor D, Factor H, Factor I, properdin, C4b-binding protein, Membrane-cofactor protein (MCB), Decay-accelerating factor (DAF), S protein and Homologous restriction factor (HRF).

Several of the immune modulators described above are FDA approved and commercially available. FDA approved interferons include interferon alfa-2a (Roferon-A®, Hoffmann-La Roche, Inc.), peginterferon alfa-a (Pegasys®, Hoffmann-La Roche, Inc.), interferon alfa-2b (Intron A®, Schering-Plough Corporation), PEGylated interferon alfa-2b (PEG-Intron™, Schering-Plough Corporation), interferon alfa-n1 (Wellferon®, GlaxoSmithKline), interferon alfa-n3 (Alferon N®, Interferon Sciences, Inc.), interferon beta-1a (Avonex®, Biogen, Inc.; and Rebif®, Serono, Inc.), interferon beta-1b (Betaseron®, Chiron Corp. and Berlex Laboratories), interferon gamma-1b (Actimmune®, Intermune Pharmaceuticals, Inc.). In addition, GM-CSF has been approved by the FDA under the tradename of Leukine® (Berlex Laboratories) and IL-2 has been approved for use under the tradename Proleukin® (Chiron Corp®).

B. Photosensitizer Compositions

Photosensitizers known in the art are typically selected for use according to: 1) efficacy in delivery, 2) proper localization in target tissues, 3) wavelengths of absorbance, 4) proper excitatory wavelength, 5) purity, and 6) in vivo effects on pharmacokinetics, metabolism, and reduced toxicity.

A photosensitizers for clinical use is optimally amphiphilic, meaning that it shares the opposing properties of being water-soluble, yet hydrophobic. The photosensitizer should be water-soluble in order to pass through the bloodstream systemically, however it should also be hydrophobic enough to pass across cell membranes. Modifications, such as attaching polar residues (amino acids, sugars, and nucleosides) to the hydrophobic porphyrin ring, can alter polarity and partition coefficients to desired levels. Such methods of modification are well known in the art.

Without being bound by theory, it is believed that photosensitizers of the present invention can bind to lipoproteins present in the bloodstream and be transported to inflammatory cells of atherosclerotic plaques. Uptake by inflammatory cells is expedited with the aid of immune modulators. As a result, photosensitizers are selectively delivered to these cells at a higher level and with faster kinetics.

In specific embodiments, photosensitizers of the present invention absorb light at a relatively long wavelength, thereby absorbing at low energy. Low-energy light can travel further through tissue than high-energy light, which becomes scattered. Optimal tissue penetration by light occurs between about 650 and about 800 nm. Porphyrins found in red blood cells typically absorb at about 630 nm, and new, modified porphyrins have optical spectra that have been “red-shifted”, in other words, absorbs lower energy light. Other naturally occurring compounds have optical spectra that is red-shifted with respect to porphyrin, such as chlorins found in chlorophyll (about 640 to about 670 nm) or bacteriochlorins found in photosynthetic bacteria (about 750 to about 820 nm).

Photosensitizers of the invention can be any known in the art, and optionally coupled to molecular carriers.

i) Porphyrins and Hydroporphyrins

Porphyrins and hydroporphyrins can include, but are not limited to, Photofrin® (porfimer sodium), hematoporphyrin IX, hematoporphyrin esters, dihematoporphyrin ester, synthetic diporphyrins, O-substituted tetraphenyl porphyrins (picket fence porphyrins), 3,1-meso tetrakis (o-propionamido phenyl) porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylenedicarboxylate adducts of benzoporphyrin, endogenous metabolic precursors, δ-aminolevulinic acid, benzonaphthoporphyrazines, naturally occurring porphyrins, ALA-induced protoporphyrin IX, synthetic dichlorins, bacteriochlorins of the tetra(hydroxyphenyl) porphyrin series, purpurins, tin and zinc derivatives of octaethylpurpurin, etiopurpurin, tin-etio-purpurin, porphycenes, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, chlorin e₆ monoethylendiamine monamide, verdins such as, but not limited to zinc methyl pyroverdin (ZNMPV), copro II verdin trimethyl ester (CVTME) and deuteroverdin methyl ester (DVME), pheophorbide derivatives, and pyropheophorbide compounds, texaphyrins with or without substituted lanthanides or metals, lutetium (III) texaphyrin, and gadolinium(III) texaphyrin.

Porphyrins, hydroporphyrins, benzoporphyrins, and derivatives are all related in structure to hematoporphyrin, a molecule that is a biosynthetic precursor of heme, which is the primary constituent of hemoglobin, found in erythrocytes. First-generation and naturally occurring porphyrins are excited at about 630 nm and have an overall low fluorescent quantum yield and low efficiency in generating reactive oxygen species. Light at about 630 nm can only penetrate tissues to a depth of about 3 mm, however there are derivatives that have been ‘red-shifted’ to absorb at longer wavelengths, such as the benzoporphyrins BPD-MA (Verteporfin). Thus, these ‘red-shifted’ derivatives show less collateral toxicity compared to first-generation porphyrins.

Chlorins and bacteriochlorins are also porphyrin derivatives, however these have the unique property of hydrogenated exo-pyrrole double bonds on the porphyrin ring backbone, allowing for absorption at wavelengths greater than about 650 nm. Chlorins are derived from chlorophyll, and modified chlorins such as meta-tetra hydroxyphenylchlorin (mTHPC) have functional groups to increase solubility. Bacteriochlorins are derived from photosynthetic bacteria and are further red-shifted to about 740 nm. A specific embodiment of the invention uses chlorinₑ₆.

Purpurins, porphycenes, and verdins are also porphyrin derivatives that have efficacies similar to or exceeding hematoporphyrin. Purpurins contain the basic porphyrin macrocycle, but are red-shifted to about 715 nm. Porphycenes have similar activation wavelengths to hematoporphyrin (about 635 nm), but have higher fluorescence quantum yields. Verdins contain a cyclohexanone ring fused to one of the pyrroles of the porphyrin ring. Phorbides and pheophorbides are derived from chlorophylls and have 20 times the effectiveness of hematoporphyrin. Texaphyrins are new metal-coordinating expanded porphyrins. The unique feature of texaphyrins is the presence of five, instead of four, coordinating nitrogens within the pyrrole rings. This allows for coordination of larger metal cations, such as trivalent lanthanides. Gadolinium and lutetium are used as the coordinating metals. Texaphyrins also tend to accumulate within atherosclerotic plaques and even so, providing texaphyrins to inflammatory cells of plaques according to methods of the present invention will increase the specificity of photoactivation. In a specific embodiment, the photosensitizer can be Antrin®, otherwise known as motexafin lutetium.

5-aminolevulinic acid (ALA) is a precursor in the heme biosynthetic pathway, and exogenous administration of this compound causes a shift in equilibrium of downstream reactions in the pathway. In other words, the formation of the immediate precursor to heme, protoporphyrin IX, is dependent on the rate of 5-aminolevulinic acid synthesis, governed in a negative-feedback manner by concentration of free heme. Conversion of protoporphyrin IX is slow, and where desired, administration of exogenous ALA can bypass the negative-feedback mechanism and result in accumulation of phototoxic levels of ALA-induced protoporphyrin IX. ALA is rapidly cleared from the body, but like hematoporphyrin, has an absorption wavelength of about 630 nm.

First-generation photosensitizers are exemplified by the porphyrin derivative Photofrin®, also known as porfimer sodium. Photofrin® is derived from hematoporphyrin-IX by acid treatment and has been approved by the Food and Drug Administration for use in PDT. Photofrin® is characterized as a complex and inseparable mixture of monomers, dimers, and higher oligomers. There has been substantial effort in the field to develop pure substances that can be used as successful photosensitizers. Thus, in a specific embodiment, the photosensitizer is a benzoporphyrin derivative (“BPD”), such as BPD-MA, also commercially known as Verteporfin. U.S. Pat. No. 4,883,790 describes BPDs. Verteporfin has been thoroughly characterized (Richter et al., 1987; Aveline et al., 1994; Levy, 1994) and it has been found to be a highly potent photosensitizer for PDT. Verteporfin has been used in PDT treatment of certain types of macular degeneration, and is thought to specifically target sites of new blood vessel growth, or angiogenesis, such as those observed in “wet” macular degeneration. Verteporfin is typically administered intravenously, with an optimal incubation time range from 1.5 to 6 hours. Verteporfin absorbs at 690 nm, and is activated with commonly available light sources. One tetrapyrrole-based photosensitizer having recent success in the clinic is MV0633 (Miravant). MV0633 is well suited for cardiovascular therapies and as such, can be used in therapeutic and diagnostic methods of the invention.

In specific embodiments, the photosensitizer has a chemical structure that includes multiple conjugated rings that allow for light absorption and photoactivation, e.g., the photosensitizer can produce singlet oxygen upon absorption of electromagnetic irradiation at the proper energy level and wavelength. Such specific embodiments include motexafin lutetium (Antrin®) and chlorinₑ₆.

ii) Cyanine and Other Photoactive Dyes

Cyanine and other dyes include but are not limited to merocyanines, phthalocyanines with or without metal substituents, chloroaluminum phthalocyanine with or without varying substituents, sulfonated aluminum PC, ring-substituted cationic PC, sulfonated AlPc, disulfonated and tetrasulfonated derivative, sulfonated aluminum naphthalocyanines, naphthalocyanines with or without metal substituents and with or without varying substituents, tetracyanoethylene adducts, nile blue, crystal violet, azure β chloride, rose bengal, benzophenothiazinium compounds and phenothiazine derivatives including methylene blue.

Cyanines are deep blue or purple compounds that are similar in structure to porphyrins. However, these dyes are much more stable to heat, light, and strong acids and bases than porphyrin molecules. Cyanines, phthalocyanines, and naphthalocyanines are chemically pure compounds that absorb light of longer wavelengths than hematoporphyrin derivatives with absorption maxima at about 680 nm. Phthalocyanines, belonging to a new generation of substances for PDT are chelated with a variety of diamagnetic metals, chiefly aluminum and zinc, which enhance their phototoxicity. A ring substitution of the phthalocyanines with sulfonated groups will increase solubility and affect the cellular uptake. Less sulfonated compounds, which are more lipophilic, show the best membrane-penetrating properties and highest biological activity. The kinetics are much more rapid than those of HPD, where, for example, high tumor to tissue ratios (8:1) were observed after 1-3 hours. The cyanines are eliminated rapidly and almost no fluorescence can be seen in the tissue of interest after 24 hours.

Other photoactive dyes such as methylene blue and rose bengal, are also used for photodynamic therapy. Methylene blue is a phenothiazine cationic dye that is exemplified by its ability to specifically target mitochondrial membrane potential. Rose-bengal and fluorescein are xanthene dyes that are well documented in the art for use in photodynamic therapy. Rose bengal diacetate is an efficient, cell-permeant generator of singlet oxygen. It is an iodinated xanthene derivative that has been chemically modified by the introduction of acetate groups. These modifications inactivate both its fluorescence and photosensitization properties, while increasing its ability to cross cell membranes. Once inside the cell, esterases remove the acetate groups and restore rose bengal to its native structure. This intracellular localization allows rose bengal diacetate to be a very effective photosensitizer.

iii) Other Photosensitizers

Diels-Alder adducts, dimethyl acetylene dicarboxylate adducts, anthracenediones, anthrapyrazoles, aminoanthraquinone, phenoxazine dyes, chalcogenapyrylium dyes such as cationic selena and tellurapyrylium derivatives, cationic imminium salts, and tetracyclines are other compounds that also exhibit photoactive properties and can be used advantageously in photodynamic therapy. Other photosensitizers that do not fall in either of the aforementioned categories have other uses besides photodynamic therapy, but are also photoactive. For example, anthracenediones, anthrapyrazoles, aminoanthraquinone compounds are often used as anticancer therapies (i.e. mitoxantrone, doxorubicin).

Chalcogenapyrylium dyes such as cationic selena- and tellurapyrylium derivatives have also been found to exhibit photoactive properties in the range of about 600 to about 900 nm range, more preferably from about 775 to about 850 nm. In addition, antibiotics such as tetracyclines and fluoroquinolone compounds have demonstrated photoactive properties.

iv) Devices and Methods for Photoactivation

Typically, administration of photosensitizers is followed by a sufficient period of time to allow accumulation of the photosensitizer at the target site. Following this period of time, the photosensitizer is activated by irradiation. This is accomplished by applying light of a suitable wavelength and intensity, for an effective length of time, at the site of the plaque. As used herein, “irradiation” refers to the use of light to induced a chemical reaction of a photosensitizer.

The suitable wavelength, or range of wavelengths, will depend on the particular photosensitizer(s) used, and can range from about 450 nm to about 550 nm, from about 550 nm to about 650 nm, from about 650 nm to about 750 nm, from about 750 nm to about 850 nm and from about 850 nm to about 950 nm.

In specific embodiments, target tissues are illuminated with red light. Given that red and/or near infrared light best penetrates mammalian tissues, photosensitizers with strong absorbances in the range of about 600 nm to about 900 nm are optimal for PDT. For photoactivation, the wavelength of light is matched to the electronic absorption spectrum of the photosensitizer so that the photosensitizer absorbs photons and the desired photochemistry can occur. Wavelength specificity for photoactivation generally depends on the molecular structure of the photosensitizer. Photoactivation can also occur with sub-ablative light doses. Determination of suitable wavelength, light intensity, and duration of illumination is within ordinary skill in the art.

The effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (about 700 to about 800 nm) (Svaasand and Ellingsen, (1983) Photochem Photobiol. 38:293-299). Altering the biologic interactions and physical characteristics of the photosensitizer can alter these values. In general, photosensitizers with longer absorbing wavelengths and higher molar absorption coefficients at these wavelengths are more effective photodynamic agents.

Photoactivating dosages depend on various factors, including the amount of the photosensitizer administered, the wavelength of the photoactivating light, the intensity of the photoactivating light, and the duration of illumination by the photoactivating light. Thus, the dose can be adjusted to a therapeutically effective dose by adjusting one or more of these factors. Such adjustments are within the level of ordinary skill in the art.

The light for photoactivation can be produced and delivered to the plaque site by any suitable means known in the art. Photoactivating light can be delivered to the plaque site from a light source, such as a laser or optical fiber. Preferably, optical fiber devices that directly illuminate the plaque site deliver the photoactivating light. For example, the light can be delivered by optical fibers threaded through small gauge hypodermic needles. Light can be delivered by an appropriate intravascular catheter, such as those described in U.S. Pat. Nos. 6,246,901 and 6,096,289, which can contain an optical fiber. Optical fibers can also be passed through arthroscopes. In addition, light can be transmitted by percutaneous instrumentation using optical fibers or cannulated waveguides. For open surgical sites, suitable light sources include broadband conventional light sources, broad arrays of light-emitting diodes (LEDs), and defocused laser beams.

Delivery can be by all methods known in the art, including transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, (e.g., lightboxes or convergent light beams).

Where treatment is desired, the dosage of photosensitizer composition, and light activating the photosensitizer composition, is administered in an amount sufficient to produce a phototoxic species. For example, where the photosensitizer is chlorinₑ₆, administration to humans is in a dosage range of about 0.5 to about 10 mg/kg, preferably about 1 to about 5 mg/kg more preferably about 2 to about 4 mg/kg and the light delivery time is spaced in intervals of about 30 minutes to about 3 days, preferably about 12 hours to about 48 hours, and more preferably about 24 hours. The light dose administered is in the range of about 20-500 J/cm, preferably about 50 to about 300 J/cm and more preferably about 100 to about 200 J/cm. The fluence rate is in the range of about 20 to about 500 mw/cm, preferably about 50 to about 300 mw/cm and more preferably about 100 to about 200 mw/cm. There is a reciprocal relationship between photosensitizer compositions and light dose, thus, determination of suitable wavelength, light intensity, and duration of illumination is within ordinary skill in the art.

In performing methods of the invention, it is desirable for the phototoxic species to induce apoptosis and not necrosis of the cells comprising the vulnerable plaque. Lowering the fluence rate will favor apoptosis (e.g., less than about 100 mw/cm, e.g., about 10 to about 60 mw/cm, for chlorinₑ₆). The wavelength and power of light can be adjusted according to standard methods known in the art to control the production of phototoxic species. Thus, under certain conditions (e.g., low power, low fluence rate, shorter wavelength of light or some combination thereof), a fluorescent species is primarily produced from the photosensitizer and any reactive species produced has a negligible effect. These conditions are easily adapted to bring about the production of a phototoxic species. For example, where the photosensitizer is chlorinₑ₆, the light dose administered to produce a fluorescent species and an insubstantial reactive species is less than about 10 J/cm, preferably less than about 5 J/cm and more preferably less than about 1 J/cm. Determination of suitable wavelength, light intensity, and duration of illumination for any photosensitizer is within the level of ordinary skill in the art.

In a specific embodiment, photoactivation can be carried out using a specially designed intravascular device that delivers excitation light to the plaque surface inside the artery and receives emitted fluorescence or other detectable signals (e.g., heat or radioactivity) that are transmitted to an analysis instrument. The same device can optionally be used to deliver therapeutic light when a fluorescent signal or other measurable signal (e.g., heat or radioactivity) is detected. Examples of such devices are provided by PCT/US02/38852, filed Dec. 3, 2003, as well as U.S. Application Publication Nos. 20030103995 (Ser. No. 10/163744, filed Jun. 4, 2002) and 20030082105 (Ser. No. 10/215958, filed Aug. 9, 2002).

C. Fluorescent Markers

Fluorescent markers of the present invention can be any known in the art, including photosensitizers, fluorescent dyes, and photoactive dyes which are optionally coupled to molecular carriers.

Fluorescent dyes of the present invention can be any known in the art, including, but not limited to 6-carboxy-4′,5′-dichloro-2′,7′-dimethoxyfluorescein succinimidyl ester; 5-(and-6)-carboxyeosin; 5-carboxyfluorescein; 6-carboxyfluorescein; 5-(and-6)-carboxyfluorescein; 5-carboxyfluorescein-bis-(5-carboxymethoxy-2-nitrobenzyl)ether, -alanine-carboxamide, or succinimidyl ester; 5-carboxyfluorescein succinimidyl ester; 6-carboxyfluorescein succinimidyl ester; 5-(and-6)-carboxyfluorescein succinimidyl ester; 5-(4,6-dichlorotriazinyl) aminofluorescein; 2′,7′-difluorofluorescein; eosin-5-isothiocyanate; erythrosin-5-isothiocyanate; 6-(fluorescein-5-carboxamido)hexanoic acid or succinimidyl ester; 6-(fluorescein-5-(and-6)-carboxamido)hexanoic acid or succinimidyl ester; fluorescein-5-EX succinimidyl ester; fluorescein-5-isothiocyanate; fluorescein-6-isothiocyanate; Oregon Green® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidyl ester or triethylammonium salt; Oregon Green® 514 carboxylic acid; Oregon Green® 514 carboxylic acid or succinimidyl ester; Rhodamine Green™ carboxylic acid, succinimidyl ester or hydrochloride; Rhodamine Green™ carboxylic acid, trifluoroacetamide or succinimidyl ester; Rhodamine Green™-X succinimidyl ester or hydrochloride; Rhodol Green™ carboxylic acid, N,O-bis-(trifluoroacetyl) or succinimidyl ester; bis-(4-carboxypiperidinyl)sulfonerhodamine or di(succinimidyl ester); 5-(and-6)-carboxynaphthofluorescein, 5-(and-6)-carboxynaphthofluorescein succinimidyl ester; 5-carboxyrhodamine 6G hydrochloride; 6-carboxyrhodamine 6G hydrochloride, 5-carboxyrhodamine 6G succinimidyl ester; 6-carboxyrhodamine 6G succinimidyl ester; 5-(and-6)-carboxyrhodamine 6G succinimidyl ester; 5-carboxy-2′,4′,5′,7′-tetrabromosulfonefluorescein succinimidyl ester or bis-(diisopropylethylammonium) salt; 5-carboxytetramethylrhodamine; 6-carboxytetramethylrhodamine; 5-(and-6)-carboxytetramethylrhodamine; 5-carboxytetramethylrhodamine succinimidyl ester; 6-carboxytetramethylrhodamine succinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green isothiocyanate; NANOGOLD® mono(sulfosuccinimidyl ester); QSY® 21 carboxylic acid or succinimidyl ester; QSY® 7 carboxylic acid or succinimidyl ester; Rhodamine Red™-X succinimidyl ester; 6-(tetramethylrhodamine-5-(and-6)-carboxamido)hexanoic acid succinimidyl ester; tetramethylrhodamine-5-isothiocyanate; tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; and X-rhodamine-5-(and-6)-isothiocyanate.

Fluorescent dyes of the present invention can be, for example, BODIPY® dyes commercially available from Molecular Probes, including, but not limited to BODIPY® FL; BODIPY® TMR STP ester; BODIPY® TR-X STP ester; BODIPY® 630/650-X STP ester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoic acid; 4,4-difluoro-5,7-dimethyl-4-bora-3a,4a-diaza-s-indacene-3-pentanoic acid succinimidyl ester; 4,4-difluoro-5,7-dimethyl-4-bora-3a,4a-diaza-s-indacene-3-propionic acid; 4,4-difluoro-5,7-dimethyl-4-bora-3a,4a-diaza-s-indacene-3-propionic acid succinimidyl ester; 4,4-difluoro-5,7-dimethyl-4-bora-3a,4a-diaza-s-indacene-3-propionic acid sulfosuccinimidyl ester or sodium salt; 6-((4,4-difluoro-5,7-dimethyl-4-bora-3a,4a-diaza-s-indacene-3-propionyl)amino)hexanoic acid; 6-((4,4-difluoro-5,7-dimethyl-4-bora-3a,4a-diaza-s-indacene-3-propionyl)amino)hexanoic acid or succinimidyl ester; N-(4,4-difluoro-5,7-dimethyl-4-bora-3a,4a-diaza-s-indacene-3-propionyl)cysteic acid, succinimidyl ester or triethylammonium salt; 6-4,4-difluoro-1,3-dimethyl-5-(4-methoxyphenyl)-4-bora-3a,4a 4,4-difluoro-5,7-diphenyl-4-bora-3a,4a-diaza-s-indacene-3-propionic acid; 4,4-difluoro-5,7-diphenyl-4-bora-3a,4a-diaza-s-indacene-3-propionic acid succinimidyl ester; 4,4-difluoro-5-phenyl-4-bora-3a,4a-diaza-s-indacene-3-propionic acid succinimidyl ester; 6-((4,4-difluoro-5-phenyl-4-bora-3a,4a-diaza-s-indacene-3-propionyl)amino)hexanoic acid or succinimidyl ester; 4,4-difluoro-5-(4-phenyl-1,3-butadienyl)-4-bora-3a,4a-diaza-s-indacene-3-propionic acid succinimidyl ester; 4,4-difluoro-5-(2-pyrrolyl)-4-bora-3a,4a-diaza-s-indacene-3-propionic acid succinimidyl ester; 6-(((4,4-difluoro-5-(2-pyrrolyl)-4-bora-3a,4a-diaza-s-indacene-3-yl)styryloxy)acetyl)aminohexanoic acid or succinimidyl ester; 4,4-difluoro-5-styryl-4-bora-3a,4a-diaza-s-indacene-3-propionic acid; 4,4-difluoro-5-styryl-4-bora-3a,4a-diaza-s-indacene-3-propionic acid succinimidyl ester; 4,4-difluoro-1,3,5,7-tetramethyl-4-bora-3a,4a-diaza-s-indacene-8-propionic acid; 4,4-difluoro-1,3,5,7-tetramethyl-4-bora-3a,4a-diaza-s-indacene-8-propionic acid succinimidyl ester; 4,4-difluoro-5-(2-thienyl)-4-bora-3a,4a-diaza-s-indacene-3-propionic acid succinimidyl ester; 6-(((4-(4,4-difluoro-5-(2-thienyl)-4-bora-3a,4a-diaza s-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester.

Fluorescent dyes the present invention can be, for example, Alexa fluor dyes commercially available from Molecular Probes, including but not limited to Alexa Fluor® 350 carboxylic acid; Alexa Fluor® 430 carboxylic acid; Alexa Fluor® 488 carboxylic acid; Alexa Fluor® 532 carboxylic acid; Alexa Fluor® 546 carboxylic acid; Alexa Fluor® 555 carboxylic acid; Alexa Fluor® 568 carboxylic acid; Alexa Fluor® 594 carboxylic acid; Alexa Fluor® 633 carboxylic acid; Alexa Fluor® 647 carboxylic acid; Alexa Fluor® 660 carboxylic acid; and Alexa Fluor® 680 carboxylic acid. Fluorescent dyes the present invention can also be, for example, cyanine dyes commercially available from Amersham-Pharmacia Biotech, including, but not limited to Cy3 NHS ester; Cy 5 NHS ester; Cy5.5 NHS ester; and Cy 7 NHS ester.

Photoactive dyes of the present invention can be any photosensitizer known in the art, which will fluoresce but not necessarily produce a reactive species in phototoxic amounts when illuminated. Depending on the wavelength and power of light administered, a photosensitizer can be activated to fluoresce and, therefore, act as a photoactive dye, but not produce a phototoxic effect unless, in some cases, the wavelength and power of light is suitably adapted to induce a phototoxic effect.

D. Radiolabeled Markers

Radiolabeled markers of the present invention can comprise any known in the art, including, but not limited to radionuclide or a paramagnetic contrast agents, preferably beta-emitting agents, which are optionally coupled to molecular carriers.

Examples of appropriate radionuclides for use in radiolabeling include, but are not limited to ¹³¹I, ¹²⁵I, ^{99m}Tc, ¹⁸F, ⁶⁸Ga, ⁶⁷Ga, ⁷²As, ⁸⁹Zr, ⁶²Cu, ¹¹¹Cu, ²⁰³In, ¹⁹⁸Pb, ¹⁹⁸Hg, ⁹⁷Ru, ¹¹C, ¹⁸⁸Re, and ²⁰¹Tl. Suitable paramagnetic contrast agents include, but are not limited to gadolinium, cobalt, nickel, manganese and iron.

In a specific embodiment, the diagnostic agent is a β-emitter, for example, ¹⁸F-Fluorodeoxyglucose (“FDG”). Other β-emitters include but are not limited to ¹³¹I, ¹⁸⁶Re, which is electron emitting, or ¹⁸⁸Re, which is positron emitting. β-detecting devices distinguish β rays from γ rays by a ratio of about 100:1 (i.e., 100:1 β to γ), more preferably by a ratio of 1000:1 (i.e, 1000:1 β to γ).

Detection of radiolabeled compositions can comprise imaging or standard means known in the art. For example, radionuclides or paramagnetic contrast agents can be detected by gamma detecting devices. One of ordinary skill in the art will appreciate that the methods of detecting positrons or γ-photons, as well as radionuclides, will require different detection techniques.

Imaging methods such as magnetic resonance imaging (MRI), and computer tomography (CT), are widely used because of their ability to non-invasively image body organs and tissues with minor deleterious effects. In these techniques, an organ or tissue is irradiated with electromagnetic waves. The waves reflected or scattered by the organ or tissue are recorded and processed into a digital image.

Generally, MRI is a well-known imaging technique. A conventional MRI device establishes a homogenous magnetic field, for example, along an axis of a person's body that is to undergo MRI. This homogeneous magnetic field conditions the interior of the person's body for imaging by aligning the nuclear spins of nuclei (in atoms and molecules forming the body tissue) along the axis of the magnetic field. For discussions on in vivo nuclear magnetic resonance imaging, see, for example, Schaefer et al., (1989) JACC 14, 472-480; Shreve et al., (1986) Magn. Reson. Med. 3, 336-340; Wolf, G. L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415.

Positron Emission Tomography (PET) and Single Photon Emission Computed Tomography (SPECT) are imaging techniques in which a radionuclide is synthetically introduced into a molecule of potential biological significance, such as a tracer. The subsequent uptake of the radiotracer is measured over time and used to obtain information about the physiological process of interest. While PET and SPECT rely on similar principles to produce their images, differences in instrumentation, radiochemistry, and experimental applications are are accounted for by differences in their respective physics of photon emission.

Unstable nuclides that possess an excess number of protons may take one of two approaches in an effort to reduce their net nuclear positivity. In one radioactive decay scheme, a proton is converted to a neutron and a particle called a positron is emitted (Hoffman, E. J., and Phelps, M. E. New York: Raven Press; 1986: 237-286; Sorenson, J. A., and Phelps, M. E. Philadelphia: W. B. Saunders; 1987). Of identical mass but opposite charge, positrons are the antimatter equivalent of electrons. When ejected from the nucleus, a positron collides with an electron, resulting in the annihilation of both particles and the release of energy. Two γ photons are produced, each of equivalent energy and opposite trajectory (generally 180° apart).

The unique spatial signature of back-to-back photon paths is exploited by PET scanners in locating the source of an annihilation event, a method known as coincidence detection (Hoffman, E. J., and Phelps, M. E. New York: Raven Press; 1986: 237-286; Links, J. M. New York: Raven Press; 1990: 37-50). PET (and SPECT) scanners employ scintillation detectors made of dense crystalline materials (e.g., bismuth germanium oxide, sodium iodide, or cesium fluoride), that capture the high-energy photons and convert them to visible light. This brief flash of light is converted into an electrical pulse by an adjacent photomultiplier tube (PMT). The crystal and PMT together make up a radiation detector. A PET camera is constructed such that opposing detectors are electronically connected. Thus, when separate scintillation events in paired detectors coincide, an annihilation event is presumed to have occurred at some point along an imaginary line between the two. This information is used to reconstruct images using the principles of computed tomography.

Isotopes that decay by electron capture and/or γ emissions can be directly detected by SPECT. Certain proton-rich radionuclides, such as ¹²³I and ^{99m}Tc, may instead capture an orbiting electron, once again transforming a proton to a neutron (Sorenson J A, and Phelps M E. Philadelphia: W. B. Saunders; 1987). The resulting daughter nucleus often remains residually excited. This meta-stable arrangement subsequently dissipates, thereby achieving a ground state and producing a single γ photon in the process. Because γ photons are emitted directly from the site of decay, no comparable theoretical limit on spatial resolution exists for SPECT. However, instead of coincidence detection, SPECT utilizes a technique known as collimation (Jaszczak R J. Boca Raton: CRC Press; (1991): 93-118). A collimator may be thought of as a lead block containing many tiny holes that is interposed between the subject and the radiation detector. Given knowledge of the orientation of a collimator's holes, the original path of a detected photon is linearly extrapolated and the image is reconstructed by computer-assisted tomography.

In a specific embodiment, intravascular β-detecting devices can be used to detect signals produced by β-emitters. An intravascular beta ray detection probe offers advantages over conventional PET imaging. First, the resolution of the probe is significantly better than PET (2 vs. 6 mm). Secondly, unlike with PET, the intravascular detection of short-range positrons is not affected by non-specific uptake of a β-emitter such as ¹⁸FDG in other tissues, such as cardiac tissues. This is attributed to the fact that the β-probe detects β-particles (which travel less than 2 mm), and therefore, myocardium-derived particles do not reach the probe. On the other hand, PET detects annihilation photons, which traverse many centimeters through tissue. Thirdly, an intravascular detector enables precise localization of VP during the same sitting as diagnostic coronary angiography. This enables the local delivery of plaque-stabilizing therapy in such a way that non-invasive techniques do not.

A β-ray detector probe, for example, can be specially designed for intravascular detection. The probe may be selectively more sensitive to positrons than gamma rays or annihilation photons, rendering it particularly suitable for the detection of coronary plaques. A computerized data acquisition system coupled to the probe can be is used to collect and display the counts.

Radiolabeled markers of the invention can be used in accordance with the methods of the invention by those of skill in the art to image plaque in the cardiovascular system of a subject. Images are generated by virtue of differences in the spatial distribution of the compositions that accumulate in the various tissues and organs of the subject. The spatial distribution of the imaging agent accumulated can be measured using devices of the present invention. Stable atheromatous plaques are evident when a less intense signal is detected, indicating the presence of tissue in which a lower concentration of a radiolabeled composition accumulates relative to the concentration of the same, which accumulates in the active atheromatous plaque and vulnerable plaque.

Alternatively, an active atheromatous plaque and vulnerable plaque can be detected as a more intense signal, indicating a region of enhanced concentration of the radiolabeled composition at the site relative to the concentration of the same which accumulates in stable atheromatous plaques. The extent of accumulation of the radiolabeled composition can be quantified using known methods for quantifying radioactive emissions. A particularly useful imaging approach to employs more than one imaging agent to perform simultaneous studies. For example, simultaneous studies of perfusion and metabolic function would allow study of coupling and uncoupling of flow of metabolism, thus facilitating determinations of tissue viability after a cardiac injury. Such determinations are useful in diagnosis of cardiac ischemia, cardiomyopathy, tissue viability, hibernating heart, and other heart abnormalities.

E. Molecular Carriers

Enhanced selectivity for active atheromatous and vulnerable plaques can be achieved by using covalent conjugates or non-covalent complexes between molecular carriers having targeting specificity for inflammatory cells or other inflammatory components located in close proximity to inflammatory cells. Accordingly, diagnostic and therapeutic compositions of the present invention can comprise diagnostic or therapeutic agents “coupled” to molecular carriers. Use of molecular carriers allows, for example, a photosensitizer to be selected according to optical and photophysical properties, without relying on the molecular structure of the photosensitizer to provide a tissue-selective effect (Hasan, T. (1992) In: B. Henderson and T. Dougherty (eds.), Photodynamic Therapy: Basic Principles and Clinical Applications. pp. 187-200: Marcel Dekker).

Generally, molecular targeting is based on two facets of molecular structure. First features of the molecular carriers such as size, charge, hydrophobicity and biodegradability can be manipulated to increase accumulation or retention in the plaque, and, second, the molecular carrier can be designed to recognize antigens, receptors or other cell type specific structures present on inflammatory cells or other inflammatory components. In specific embodiments, the molecular carrier comprises serum proteins including receptor ligands (Hamblin et al. (1994) J. Photochem. Photobiol. 26:147-157; Hamblin and Newman (1994) J. Photochem. Photobiol. 26:45-56), microspheres (Bachor et al. (1991) Proc. Natl. Acad. Sci. U.S.A. 88:1580-1584), liposomes (Polo et al. (1996) Cancer Lett. 109:57-61), polymers (Hamblin et al. (1999) Br. J. Cancer 81:261-268), monoclonal antibodies (Hamblin et al. (2000) Br. J. Cancer 83:1544-1551), growth factors (Gijsens and De Witte (1998) Int. J. Oncol. 13:1171-1177), peptides (Krinick, (1994) J. Biomater. Sci. Polym. Ed. 5: 303-324), hormones (Akhlynina et al. (1995) Cancer Res. 55:1014-1019) and lipoproteins (Schmidt-Erfurth et al. (1997) Br. J. Cancer 75:54-61).

In a specific embodiment, therapeutic and diagnostic agents of the present invention are coupled to molecular carriers comprising ligands that bind to “scavenger receptors.” Scavenger receptors are membrane proteins expressed on the surface of macrophages, monocytes, endothelial cells and smooth muscle cells that recognize a wide range of ligands, both naturally occurring and synthetic (Freeman et al. (1997) Curr. Opin. Hematol. 4:41-47). Presently, there are six members of the scavenger receptor family belonging to three classes (e.g., class A, B or C). After initial binding to the scavenger receptor, the ligands are rapidly internalized and are routed to lysosomes for degradation by proteases and other lysosomal enzymes. The wide and diverse range of structures recognized by these receptors has led to them being termed “molecular flypaper” (Krieger et al. (1992) Trends Biochem. Sci. 17:141-146, 1992). The ligands are all molecules with a pronounced anionic charge that have some common conformational features (Haberland and Fogelman (1985) Proc. Natl. Acad. Sci. U.S.A. 82:2693-2697; Takata (1989) Biochem. Biophys. Acta. 984:273-280). Specific targeting of compositions to J774 and other macrophage-like cells in vitro has been achieved with conjugates of maleylated albumin, daunorubicin and doxorubicin (Mukhopadhyay et al (1992) Biochem J. 284:237-241; Basu et al. (1994) FEBS Lett. 342:249-254; Hamblin et al. (2000) Photochem Photobiol. 4:533-540).

Numerous scavenger receptor ligands known in the art (either with or without polyethyl glycolization) can be used to localize therapeutic and diagnostic compositions of the present invention to active atheromatous and vulnerable plaques, including, but not limited to glucose analogs (e.g. fluorodeoxyglucose), chemotactic peptide receptor agonist anologs, maleylated albumin, oxidized low density lipoprotein, acetylated low density lipoprotein, oxidized high density lipoprotein, lipopolysaccharide, malondialdehyde treated proteins, lipotechoic acid, formaldehyde treated albumin, glycated albumin, polyinosinic acid, glycated lipoproteins, dextran sulfate, anionic phospholipids (phosphatidylserine), fucoidin, carrageenan, polyvinyl sulfate, monoclonal antibodies that recognize CD11b or c, CD13, CD14, CD16a, CD32, or CD68, polyvinyl sulfate, crocidolite asbestos.

In a specific embodiment, therapeutic and diagnostic agents of the present invention are coupled to molecular carriers that target macrophages and/or monocytes of active atheromatous and vulnerable plaques. These molecular carriers can be targeted to, for example, tenascin C, tissue factor, tissue inhibitor of MMP 1 and 2, oxidized LDL receptor (also known in the art as CD36), heme oxygenase-1, human cartilage gp-39, IL-6, IL-6 receptor, IL-10, IL-10 receptor, lectin-like oxidized LDL-receptor (“LOX-1”), bacterial chemotactic peptide receptor agonists, preferably Formyl-Methionine-Leucine-Phenylalanine (“F-MLP”), macrophage chemoattractant protein-i receptor (“CCR-9”) and monocyte inflammatory protein-1 and receptors thereof (including “CCR-5”). Such molecular carriers can be, for example, antibodies against these biomolecules, ligands binding the same or analogs thereof.

In a specific embodiment, therapeutic and diagnostic agents of the present invention are coupled to molecular carriers that target T cells of active atheromatous and vulnerable plaques. These molecular carriers can be targeted to, for example, IL-10, IL-10 receptor, monocyte inflammatory protein-1 and receptors thereof and transferrin. Such molecular carriers can be, for example, antibodies against these biomolecules, ligands binding the same or analogs thereof, including, but not limited to monoclonal antibodies that recognize CD1, CD2, CD3, CD4, CD5, CD6, CD7, CD8, CD25, CD28, CD44, CD71 or transferrin.

In a specific embodiment, therapeutic and diagnostic agents of the present invention are delivered via molecular carriers that target the lipid pool of the atheroma, including but not limited to hydrophobic photosensitizers or photosensitizers delivered in hydrophobic vehicles such as liposomes (with positive, neutral or negatively charged and optionally containing cholesterol or cardiolipin) cremaphor EL, PEG/solvent mixtures, iodized castor oil, and various nanoparticles and micellar preparations.

In a specific embodiment, therapeutic and diagnostic agents of the present invention are coupled to molecular carriers that target proteases that degrade extracellular matrix (e.g., metalloproteinases), including but not limited to monoclonal antibodies against the protease and proteinase substrates.

In a specific embodiment, therapeutic and diagnostic agents of the present invention are coupled to molecular carriers that target the endothelial cells of active atheromatous and vulnerable plaques. These molecular carriers can be targeted to, for example, endothelial adhesion molecules including, but not limited to, ICAM (also known in the art as CD54) and VCAM (also known in the art as CD106), angiotensin II, angiotensin converting enzyme (also known in the art as CD143), endothelial derived lipase, tissue factor, heme oxygenase-1, LOX-1, low density lipoprotein (“LDL”), high density lipoprotein, (“HDL”), P-selectin, L-selectin and E-selectin. Such molecular carriers can be, for example, antibodies against these biomolecules, ligands binding the same or analogs thereof. Therapeutic and diagnostic compositions of the present invention can be coupled to molecular carriers that target the subendothelial matrix of active atheromatous and/or vulnerable plaques.

In a specific embodiment, therapeutic and diagnostic agents of the present invention are coupled to molecular carriers that target neutrophils of active atheromatous and vulnerable plaques. These molecular carriers can be targeted to, for example, myeloperoxidase. Such molecular carriers can be, for example, antibodies against these biomolecules, ligands binding the same or analogs thereof.

In a specific embodiment, therapeutic and diagnostic agents of the present invention are coupled to molecular carriers that target B cells of active atheromatous and vulnerable plaques. These molecular carriers can be targeted to, for example, IL-6, IL-6 receptor, IL-10 and IL-10 receptor. Such molecular carriers can be, for example, antibodies against these biomolecules, ligands binding the same or analogs thereof.

In a specific embodiment, therapeutic and diagnostic agents of the present invention are coupled to molecular carriers that target smooth muscle cells of active atheromatous and vulnerable plaques. These molecular carriers can be targeted to, for example, LOX-1. Such molecular carriers can be, for example, antibodies against these biomolecules, ligands binding the same or analogs thereof.

In a specific embodiment, therapeutic and diagnostic agents of the present invention are coupled to molecular carriers that either directly or indirectly associate with the target. For example, indirect targeting can be achieved by first localizing a biotinylated molecular carrier to a target, followed by administration of a streptavidin-linked composition comprising, for example, a photoactive dye, fluorescent dye, photosensitizer or radioactive agent.

Thus, localizing a therapeutic or diagnostic composition to activated macrophages or proteases that degrade extracellular matrix via a molecular carrier, for example, confers a selective advantage on an active atheromatous and vulnerable plaque, such that uptake of the composition is far greater than in a stable atheromatous plaque.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Compositions of the present invention that are useful for detection of active atheromatous and vulnerable plaques can include radiolabeled molecular carriers. A number of radiolabeled molecular carriers have been tested for their ability to bind to and permit scintigraphic detection of atherothrombotic materials. These include labeled antibodies to oxidized LDL, fibrinogen, autologous platelets, fibrin fragment E1, plasminogen activators, and ^{99m}Tc-conjugated antibodies against modified LDL (Tsimikas et al. (1999) J. Nucl. Cardiol. 6: 41-53).

Such radiolabels can be associated with the molecular carrier by ionic association or covalent bonding directly to an atom of the carrier. The radiolabel can be non-covalently or covalently associated with the carrier through a chelating structure. A “chelating structure” refers to any molecule or complex of molecules that bind to both the label and targeting moiety. Many such chelating structures are known in the art. Chelating structures include, but are not limited to —N₂S₂, —NS₃, —N₄, dota derivatives [1,4,7,10-tetrakis(carboxymethyl)-1,4,7,10-tetrazacyclododecane], an isonitrile, a hydrazine, a HYNIC (hydrazinonicotinic acid), 2-methylthiolnicotinic acid, phosphorus, or a carboxylate containing group; or through an auxiliary molecule such as mannitol, gluconate, glucoheptonate, tartrate, and the like. In some cases, chelation can be achieved without including a separate chelating structure, because the radionuclide chelates directly to atom(s) in the molecular carrier, for example to oxygen atoms in various moieties.

The chelating structure, auxiliary molecule, or radionuclide may be placed in spatial proximity to any position of the molecular carrier that does not interfere with the interaction of the targeting molecule with its target site in cardiovascular tissue. Accordingly, the chelating structure, auxiliary molecule, or radionuclide may be covalently or non-covalently associated with any moiety of the molecular carrier (except the receptor-binding moiety where the molecular carrier is a receptor and the epitope-binding region where the molecular carrier is an antibody).

Radionuclides can be placed in spatial proximity to the molecular carrier using known procedures that effect or optimize chelation, association, or attachment of the specific radionuclide to ligands. For example, when ¹²³I is the radionuclide, the imaging agent may be labeled in accordance with the known radioiodination procedures such as direct radioiodination with chloramine T, radioiodination exchange for a halogen or an organometallic group, and the like. When the radionuclide is ^{99m}Tc, the imaging agent may be labeled using any method suitable for attaching ^{99m}Tc to a ligand molecule. Preferably, when the radionuclide is ^{99m}Tc, an auxiliary molecule such as mannitol, gluconate, glucoheptonate, or tartrate is included in the labeling reaction mixture, with or without a chelating structure. More preferably, ^{99m}Tc is placed in spatial proximity to the carrier by reducing ^{99m}TcO₄, with tin in the presence of mannitol and the targeting molecule. Other reducing agents, including tin tartrate or non-tin reductants such as sodium dithionite, may also be used to make radiolabeled compositions of the present invention.

In general, labeling methodologies vary with the choice of radionuclide and the carrier to be labeled. Labeling methods are described, for example, in Peters et al. (1986) Lancet 2:946-949; Srivastava et al. (1984) Semin. Nucl. Med 14:68-82; Eckelman and Richards (1972) J. Nucl. Med. 13:180; McAfee et al. (1976) J. Nucl. Med. 17:480-487; Welch et al., (1977) J. Nucl. Med. 18:558-562; Thakur et al. (1984) Semin. Nucl. Med. 14:107; Danpure et al. (1981) Br. J. Radiol. 54:597-601; Danpure et al. (1982) Br. J. Radiol. 55:247-249; Peters et al. (1983) J. Nucl. Med. 24:39-44; Gunter et al. (1983) Radiology 149:563-566 and Thakur et al. (1985) J. Nucl. Med. 26:518-523.

After the labeling reaction is complete, the reaction mixture may optionally be purified using one or more chromatography steps such as Sep Pak or high performance liquid chromatography (HPLC). Any suitable HPLC system may be used if a purification step is performed, and the yield of cardiovascular imaging agent obtained from the HPLC step may be optimized by varying the parameters of the HPLC system, as is known in the art. Any HPLC parameter may be varied to optimize the yield of the cardiovascular imaging agent of the invention. For example, the pH may be varied, e.g., raised to decrease the elution time of the peak corresponding to the radiolabeled carrier.

Photosensitizers can also be coupled to a molecular carrier, such as a scavenger receptor ligand, either directly or indirectly via a “backbone” or “bridge” moiety, such as a polyamino acid, whereby the backbone is coupled both to the photosensitizer and the molecular carrier.

Inclusion of a backbone in a composition with a photosensitizer and a molecular carrier can provide a number of advantages, including the provision of greater stoichiometric ranges of photosensitizer and molecular carriers coupled per backbone. If the backbone possesses intrinsic affinity for a target organism, coupling to the backbone can enhance the affinity of the composition. Coupling two or more different molecular carriers to a single photosensitizer-backbone composition can expand the specific range of cells that can be targeted with one composition.

Peptides useful in the methods of the invention for design and characterization of backbone moieties include poly-amino acids which can be homo- and hetero-polymers of L-, D-, racemic DL- or mixed L- and D-amino acid composition, and which can be of defined or random mixed composition and sequence. These peptides can be modeled after particular natural peptides, and optimized by the technique of phage display and selection for enhanced binding to a chosen target, so that the selected peptide of highest affinity is characterized and then produced synthetically. Further modifications of functional groups can be introduced for purposes, for example, of increased solubility, decreased aggregation, and altered extent of hydrophobicity. Examples of nonpeptide backbones include nucleic acids and derivatives of nucleic acids such as DNA, RNA and peptide nucleic acids; polysaccharides and derivatives such as starch, pectin, chitins, celluloses and hemi-methylated celluloses; lipids such as triglyceride derivatives and cerebrosides; synthetic polymers such as polyethylene glycols (PEGS) and PEG star polymers; dextran derivatives, polyvinyl alcohols, N-(2-hydroxypropyl)-methacrylamide copolymers, poly (DL-glycolic acid-lactic acid); and compositions containing elements of any of these classes of compounds.

Modifying the charge of a component of the composition can refine the affinity of a photosensitizer composition. Conjugates such as poly-L-lysine chlorinₑ₆ can be made in varying sizes and charges (cationic, neutral, and anionic), for example, free NH2 groups of the polylysine are capped with acetyl, succinyl, or other R groups to alter the charge of the final composition. Net charge of a composition of interest can be determined by isoelectric focusing (IEF). This technique uses applied voltage to generate a pH gradient in a non-sieving acrylamide or agarose gel by the use of a system of ampholytes (synthetic buffering components). When charged polypeptides are applied to the gel they will migrate either to higher pH or to lower pH regions of the gel according to the position at which they become uncharged and hence unable to move further. This position can be determined by reference to the positions of a series of known IEF marker proteins.

In a specific embodiment, diagnostic and therapeutic compositions of the present invention can comprise diagnostic and therapeutic agents coupled molecular carriers that are antibodies. For example, photosensitizers coupled to antibodies are known in the art as “photoimmunoconjugates.” The antibody component of the composition can bind with specificity to an epitope present on the surface of an inflammatory cell associated withan active atheromatous or vulnerable plaque. As used herein, the term “binding with specificity” means that the antibody only poorly recognizes cells that do not express the epitope.

The term “antibody” as used herein includes intact molecules as well as fragments thereof, such as Fab and Fab′, which are capable of binding the epitopic determinant. Fab fragments retain an entire light chain, as well as one-half of a heavy chain, with both chains covalently linked by the carboxy terminal disulfide bond. Fab fragments are monovalent with respect to the antigen-binding site. Antibodies that can be used in the methods of the present invention can comprise whole native antibodies, bispecific antibodies; chimeric antibodies; Fab, Fab′, single chain variable region fragments (scFv) and fusion polypeptides. Preferably, the antibodies are monoclonal.

Antibodies can be prepared in several ways. Methods of producing and isolating whole native antibodies, bispecific antibodies; chimeric antibodies; Fab, Fab′, single chain V region fragments (scFv) and fusion polypeptides are known in the art. See, for example, Harlow and Lane (1988) Antibodies: A Laboratory Manual, Cold Spring Harbor Laboratory, New York (Harlow and Lane, 1988).

Antibodies are most conveniently obtained from hybridoma cells engineered to express an antibody. Methods of making hybridomas are well known in the art. The hybridoma cells can be cultured in a suitable medium, and spent medium can be used as an antibody source. Polynucleotides encoding the antibody can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition, e.g., Pristane.

Another method of obtaining antibodies is to immunize suitable host animals with an antigen and to follow standard procedures for polyclonal or monoclonal production. Monoclonal antibodies (Mabs) thus produced can be “humanized” by methods known in the art. Examples of humanized antibodies are provided, for instance, in U.S. Pat. Nos. 5,530,101 and 5,585,089.

“Humanized” antibodies are antibodies in which at least part of the sequence has been altered from its initial form to render it more like human immunoglobulins. In one version, the heavy chain and light chain C regions are replaced with human sequence. In another version, the CDR regions comprise amino acid sequences for recognition of antigen of interest, while the variable framework regions have also been converted to human sequences. See, for example, EP 0329400. In a third version, variable regions are humanized by designing consensus sequences of human and mouse variable regions, and converting residues outside the CDRs that are different between the consensus sequences. The invention encompasses humanized Mabs.

The invention also encompasses hybrid antibodies, in which one pair of heavy and light chains is obtained from a first antibody, while the other pair of heavy and light chains is obtained from a different second antibody. Such hybrids may also be formed using humanized heavy and light chains.

Construction of phage display libraries for expression of antibodies, particularly the Fab or scFv portion of antibodies, is well known in the art (Heitner et al. (2001) J Immunol Methods 248:17-30). The phage display antibody libraries that express antibodies can be prepared according to the methods described in U.S. Pat. No. 5,223,409 incorporated herein by reference. Procedures of the general methodology can be adapted using the present disclosure to produce antibodies of the present invention. The method for producing a human monoclonal antibody generally involves (1) preparing separate heavy and light chain-encoding gene libraries in cloning vectors using human immunoglobulin genes as a source for the libraries, (2) combining the heavy and light chain encoding gene libraries into a single dicistronic expression vector capable of expressing and assembling a heterodimeric antibody molecule, (3) expressing the assembled heterodimeric antibody molecule on the surface of a filamentous phage particle, (4) isolating the surface-expressed phage particle using immunoaffinity techniques such as panning of phage particles against a preselected antigen, thereby isolating one or more species of phagemid containing particular heavy and light chain-encoding genes and antibody molecules that immunoreact with the preselected antigen.

Linking light and heavy chain variable regions by using a short linking peptide makes single chain variable region fragments. Any peptide having sufficient flexibility and length can be used as a linker in a scFv. Usually the linker is selected to have little to no immunogenicity. An example of a linking peptide is (GGGGS)₃, which bridges approximately 3.5 nm between the carboxy terminus of one variable region and the amino terminus of another variable region. Other linker sequences can also be used. All or any portion of the heavy or light chain can be used in any combination. Typically, the entire variable regions are included in the scFv. For instance, the light chain variable region can be linked to the heavy chain variable region. Alternatively, a portion of the light chain variable region can be linked to the heavy chain variable region or a portion thereof. Also contemplated are compositions comprising a biphasic scFv in which one component is a polypeptide that recognizes an antigen and another component is a different polypeptide that recognizes a different antigen, such as a T cell epitope.

ScFvs can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect or mammalian cells, or prokaryotic, such as Escherichia coli, and the protein expressed by the polynucleotide can be isolated using standard protein purification techniques.

A particularly useful system for the production of scFvs is plasmid pET-22b(+) (Novagen, Madison, Wis.) in E. coli. pET-22b(+) contains a nickel ion binding domain consisting of 6 sequential histidine residues, which allows the expressed protein to be purified on a suitable affinity resin. Another example of a suitable vector is pcDNA3 (Invitrogen, San Diego, Calif.), described above.

Expression conditions should ensure that the scFv assumes functional and, preferably, optimal tertiary structure. Depending on the plasmid used (especially the activity of the promoter) and the host cell, it may be necessary or useful to modulate the rate of production. For instance, use of a weaker promoter, or expression at lower temperatures may be necessary or useful to optimize production of properly folded scFv in prokaryotic systems; or, it may be preferable to express scFv in eukaryotic cells. Antibody purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Therapeutic and diagnostic agents can be linked to antibodies according to any method known in the art. For example, the antibody can be directly linked to the agent through a polymer or a polypeptide linkage. Polymers of interest include, but are not limited to polyamines, polyethers, polyamine alcohols, derivitized to components by means of ketones, acids, aldehydes, isocyanates or a variety of other groups. Polypeptide linkages can comprise, for example poly-L-lysine linkages (Del Governatore et al. (2000) Br. J. Cancer 82:56-64; Hamblin et al. (2000) Br. J. Cancer 83:1544-41; Molpus et al. (2000) Gynecol Oncol 76:397-404). IN a specific embodiment, the antibody can be linked to a photosensitizer and at least one solubilizing agent each of which are independently bound to the antibody through a direct covalent linkage. The direct covalent linkage can be, for example, an amide linkage to a lysine residue of the antibody, as described in U.S. application number 20020197262 (Ser. No. 10/137,029; published May 1, 2002), the contents of which are herein incorporated by reference.

### III. Administration

An “effective amount” of a therapeutic composition, diagnostic composition, or immune modulator is an amount sufficient to effect a beneficial or desired clinical result. An effective amount can be administered in one or more doses. In terms of treatment, an effective amount is an amount that is sufficient to palliate, ameliorate, stabilize, reverse or slow the progression of a cardiovascular disease characterized by the presence of vulnerable plaques or otherwise reduce the pathological consequences of the impending rupture. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, and the severity of the condition.

Suitable dosages and formulations of immune modulators can be empirically determined by the administering physician. Standard texts, such as Remington: The Science and Practice of Pharmacy, 17th edition, Mack Publishing Company, and the Physician's Desk Reference, each of which are incorporated herein by reference, can be consulted to prepare suitable compositions and doses for administration. A determination of the appropriate dosage is within the skill of one in the art given the parameters for use described herein.

In accordance with the invention, “an effective amount of the radiolabeled composition” of the invention is defined as an amount sufficient to yield an acceptable signal using equipment that is available for clinical use. An effective amount of the radiolabeled composition of the invention can be administered in more than one dose. Effective amounts of the radiolabeled composition of the invention will vary according to factors such as the degree of susceptibility of the individual, the age, sex, and weight of the individual, idiosyncratic responses of the individual, and the dosimetry. Effective amounts of the imaging agent of the invention will also vary according to instrument and film-related factors. Optimization of such factors is well within the level of skill in the art. In general, the effective amount will be in the range of from about 0.1 to about 10 mg by injection or from about 5 to about 100 mg orally.

Radiolabeled markers of the present invention, optionally coupled to molecular carriers or molecular carriers and photosensitizers, can comprise, for example, from about 1 to about 30 mCi of the radionuclide in combination with a pharmaceutically acceptable carrier. Such compositions may be provided in solution or in lyophilized form. Suitable sterile and physiologically acceptable reconstitution media include water, saline, buffered saline, and the like. Radionuclides can be combined with the unlabeled molecular carrier/chelating agent and a reducing agent for a sufficient period of time and at a temperature sufficient to chelate the radionuclide to the molecular carrier prior to injection into the patient.

The radiolabeled compositions, optionally comprising molecular carriers or molecular carriers and photosensitizers, can be administered to a subject in accordance with any means that facilitates accumulation of the agent in a subject's cardiovascular system. For example, the radiolabeled composition of the invention is administered by arterial or venous injection, and has been formulated as a sterile, pyrogen-free, parenterally acceptable aqueous solution. The preparation of such parenterally acceptable solutions, having due regard to pH, isotonicity, stability, and the like, is within the skill in the art. A typical formulation for intravenous injection contains an isotonic vehicle such as Sodium Chloride Injection, Ringer's Injection, extrose Injection, Dextrose and Sodium Chloride Injection, Lactated Ringer's Injection, or other vehicle as known in the art.

The amount of radiolabeled composition used for diagnostic purposes and the duration of the study will depend upon the nature and severity of the condition being treated, on the nature of therapeutic treatments which the patient has undergone, and on the idiosyncratic responses of the patient. Ultimately, the attending physician will decide the amount of radiolabeled composition to administer to each individual patient and the duration of the imaging study.

The dosage of fluorescent markers or photosensitizers can range from about 0.1 to about 10 mg/kg. Methods for administering fluorescent compositions are known in the art, and are described, for example, in U.S. Pat. Nos. 5,952,329, 5,807,881, 5,798,349, 5,776,966, 5,789,433, 5,736,563, 5,484,803 and by (Sperduto et al. (1991) Int. J. Radiat. Oncol. Biol. Phys. 21:441-6; Walther et al. (1997) Urology 50:199-206). Such dosages may vary, for example, depending on whether multiple administrations are given, tissue type and route of administration, the condition of the individual, the desired objective and other factors known to those of skill in the art. Where the fluorescent compositions comprises a photosensitizer conjugated to an antibody, or a “photoimmunoconjugate,” dosages can vary from about 0.01 mg/m² to about 500 mg/m², preferably about 0.1 mg/m² to about 200 mg/m², most preferably about 0.1 mg/m² to about 10 mg/m². Ascertaining dosage ranges is well within the skill of one in the art. For instance, the concentration of scFv typically need not be as high as that of native antibodies in order to be therapeutically effective. Administrations can be conducted infrequently, or on a regular weekly basis until a desired, measurable parameter is detected, such as diminution of disease symptoms. Administration can then be diminished, such as to a biweekly or monthly basis, as appropriate.

Following administration of the diagnostic composition, it can be necessary to wait for the composition to reach an effective tissue concentration at the site of the plaque before detection. Duration of the waiting step varies, depending on factors such as route of administration, location, and speed of movement in the body. In addition, where the compositions are coupled to molecular carriers, the rate of uptake can vary, depending on the level of receptor expression on the surface of the cells. For example, where there is a high level of receptor expression, the rate of binding and uptake is increased. Determining a useful range of waiting step duration is within the level of ordinary skill in the art and may be optimized by utilizing fluorescence optical imaging techniques.

Compositions of the present invention are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with other pharmaceutical agents. Therapeutic compositions of photosensitizers are often administered by injection or by gradual perfusion.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow-release or sustained release forms, whereby a relatively consistent level of the active compound are provided over an extended period.

Another method of administration is intravascular, for instance by direct injection into the blood vessel, plaque or surrounding area.

Further, it may be desirable to administer the compositions locally to the area in need of treatment; this can be achieved, for example, by local infusion during surgery, by injection, by means of a catheter, or by means of an implant, said implant being of a porous, non-porous, or gelatinous material, including membranes, such as silastic membranes, or fibers. A suitable such membrane is Gliadel® provided by Guilford Pharmaceuticals Inc.

The present invention is additionally described by way of the following illustrative, non-limiting Examples that provide a better understanding of the present invention and of its many advantages.

### EXAMPLES

### Example 1

### FDG-PET and Intravascular Catheter Imaging in Atherosclerotic Plaques

Male New Zealand white rabbits ranging from 2.5 to 3.0 kilograms in weight (Charles River Breeding Lab) were maintained on a 2% cholesterol-6% peanut oil diet (ICN) for 10 weeks. After 1 week on the peanut oil diet, the abdominal aorta was denuded of endothelium by a modified Baumgartner technique. Briefly, each animal was anesthetized with a mixture of ketamine and xylazine and the right femoral artery was isolated. Subsequently, a 4 F Fogarty embolectomy catheter was introduced via arteriotomy and advanced under fluoroscopic guidance to the level of the diaphragm. The balloon was then inflated to 3 psi (pounds per square inch) above balloon inflation pressure and three passes were made down the abdominal aorta with the inflated catheter. The femoral artery was subsequently ligated and the wound closed. This animal model system is standardly used in the art for the study of active atheromatous and vulnerable plaques.

Additionally, FDG was administered intravenously to two additional rabbits. FDG accumulates in vulnerable plaques. Thereafter, a 1.6 mm thin, flexible beta probe, was inserted into the aorta. This probe is selectively sensitive to positron emissions, and was built by optically coupling a 1 mm diameter, 2 mm-long plastic scintillator to a PMT via a 40 cm long optical fiber. Measurements of FDG activity were made in triplicate, at 2 seconds per measurement, at grossly visible sites of plaque within areas of balloon injury; at non-injured sites in the cholesterol fed rabbits; and in corresponding areas in control aorta. Aortic segments at previously assessed sites of plaque and control areas were excised and examined for uptake of FDG by standard well counting.

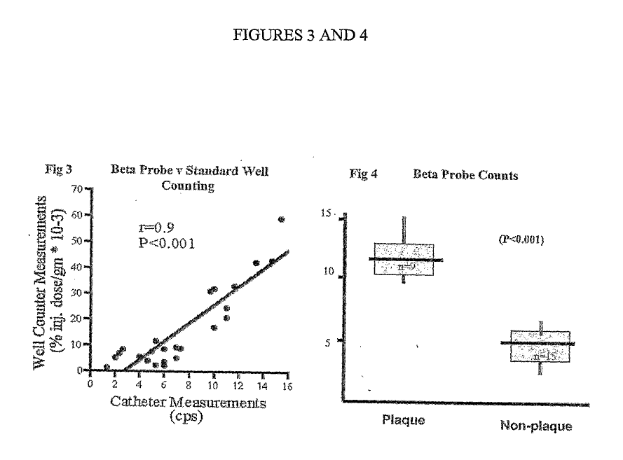

Sensitivity of the probe was 850±11 cps/μCi (mean±SD). Catheter-determined activity correlated well with well counting measurements, (r=0.89, P<0.001, FIG. 3). Moreover, atherosclerotic regions were readily distinguished from control by catheter mounted beta probe, (11.9±2.1 [n=9, range 9.7±15.3] vs. 4.8±1.9[n=14, range 1.3±7.3], cps in atherosclerotic vs. control regions, respectively, P<0.001; FIG. 4).

Intravascular detection of positron emissions was achieved with sensitivity and specificity in this in vivo system.

### Example 2

### Accumulation of ^{99m}Tc-Chemotactic Peptide in Rabbit Aorta

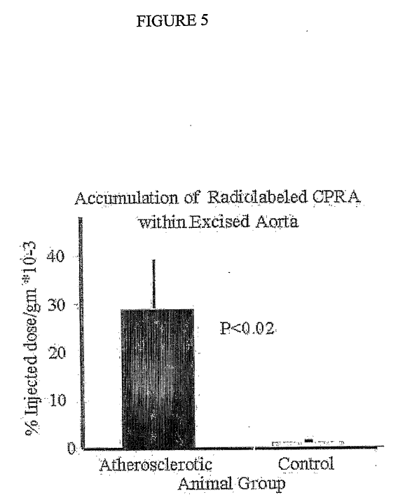

Nuclear methods of targeting inflammatory cells can detect macrophage-rich atherosclerotic lesions. An animal model of atherosclerosis was generated in which macrophage-rich atherosclerotic plaques were induced in New Zealand rabbits by balloon de-endothelialization of the infra-diaphragmatic aorta followed by a high cholesterol diet. At 10 weeks, a ^{99m}technetium radiolabeled derivative of bacterial chemotactic peptide CPRA was administered to 7 control rabbits, and to 7 rabbits in which aortic atherosclerotic lesions were induced. This peptide has been shown to bind avidly to leukocytes, with high specificity (Babich, J. W. et al, 1997). At 12 hours after the administration of the radiolabel, the live rabbits were imaged using single photon emission tomography (SPECT). Two investigators that were blinded to the status of the rabbits examined the images. A semi-quantitative scoring system was employed, in which a score of 0 (no uptake) to 3 (high uptake) was assigned to each rabbit's aortic image. At 16 hours, the animals were sacrificed and the aortas were examined for the uptake of radiolabeled ligand. Average radiotracer uptake within the atherosclerotic aortas was 72-fold higher than in the control aortas (See FIG. 5; 297±187 vs. 4±1, P=<0.02). Moreover, with SPECT imaging of the live rabbits, the mean score for radiotracer uptake was significantly higher in the atherosclerotic aortas compared to healthy aortas (2.5±0.3 vs. 0.3±0.1, P=<0.03). These data depict differential imaging between inflamed vs healthy aortic tissue and support the strategy of employing methods of the invention to detect macrophage-rich vulnerable plaque.

### Example 3

### Preparation and Purification of Photosensitizer Compounds

A photosensitizer composition comprising chlorinₑ₆ (“cₑ₆”) coupled to maleylated-albumin) was prepared for targeting to macrophages of a vulnerable plaque animal model system.

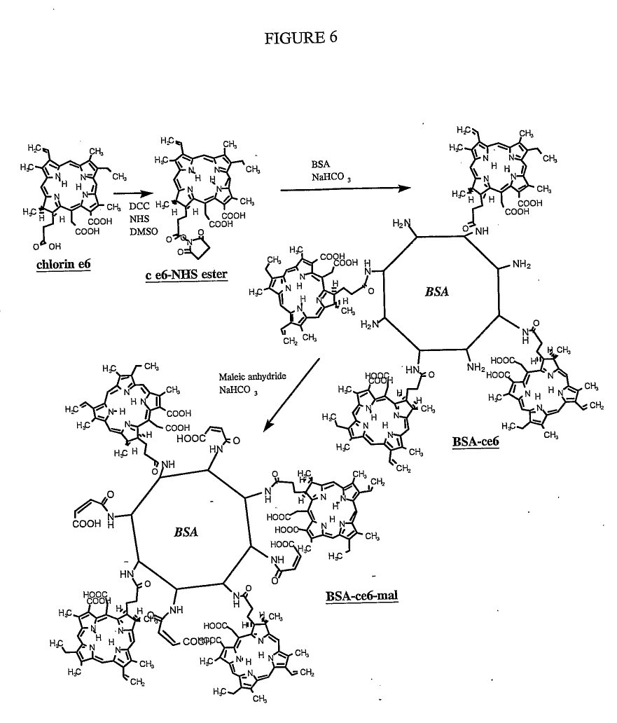

Four photosensitizer compositions were studied (i.e., two BSA-cₑ₆ conjugates and their maleylated counterparts). The N-hydroxy succinimide (NHS) ester of cₑ₆ was prepared by reacting approximately 1.5 equivalents of dicyclohexylcarbodiimide and approximately 1.5 equivalents of NHS with approximately 1 equivalent of cₑ₆ (Porphyrin Products, Logan, Utah) in dry DMSO. After standing in the dark at room temperature for approximately 24 hours, the NHS ester was frozen in aliquots for further use. BSA (Sigma Chemical Co, St Louis, Mo.) (approximately 2×50 mg) was dissolved in NaHCO₃ buffer (0.1 M, pH 9.3, approximately 3 ml), and approximately 30 μl and approximately 120 μl of cₑ₆-NHS ester added to respective tubes with vortex mixing. After standing in the dark at room temperature for approximately 6 hours, the crude conjugate preparations were each divided into two approximately equal parts. One portion of each of the conjugate preparations-was maleylated by adding solid maleic anhydride (approximately 20 mg) to the protein preparation in portions and with vortex mixing, and by adding saturated NaHCO₃ solution as needed to keep the pH above approximately 7.0 (Takata et al. (1989) Biochim. Biophys. Acta 984:273). The reaction mixture was allowed to stand at room temp in the dark for approximately 3 hours (FIG. 6). Unmodified BSA was also maleylated to act as a control and as a competitor for the cellular uptake of conjugates.

Crude conjugate preparations (approximately 5 mg/ml) were added to approximately 10× volume of acetone (ACS grade) slowly at approximately 4° C., and were kept at approximately 4° C. for approximately 6 hours, followed by centrifugation at about 4000×g for approximately 15 minutes at about 4° C. The supernatant was removed and the pellet again suspended in approximately the same volume of acetone and the centrifugation repeated. After each precipitation step the preparation was monitored by thin layer chromatography (TLC). Approximately five precipitation steps were necessary to completely remove non-covalently bound chlorin species. Finally, the pellet was dissolved in approximately 2 ml PBS and dialyzed approximately twice against 20 L PBS overnight to remove traces of acetone.

Sephadex G50 column chromatography was carried out by applying the reaction mixture from conjugation of approximately 50 mg BSA with approximately 5 mg cₑ₆-NHS ester to a 50×1 cm Sephadex column that was eluted with PBS at about 4° C. The absorbance of the eluted fractions was monitored at 400 nm and at 280 nm.

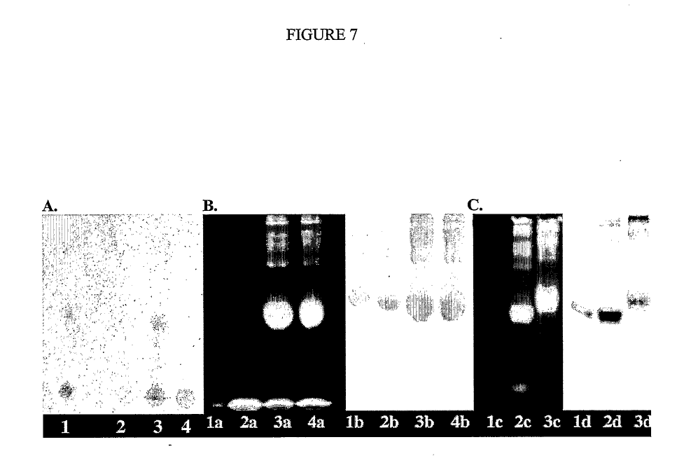

The conjugates were purified using an acetone precipitation that allowed the lipophilic cₑ₆ species to be retained in the acetone supernatant and the precipitated conjugates to be redissolved in a purified form. The sodium dodecyl sulfate polyacrylamide (SDS-PAGE) gels were viewed by fluorescence imaging to localize the cₑ₆ after staining with Coomassie Blue. FIG. 7 shows the corresponding fluorescence and Coomassie images of BSA, BSA mixed with free cₑ₆ and conjugates (BSA-cₑ₆ 1 and mal-BSA-cₑ₆ 1) after Sepahadex column chromatography, but before acetone precipitation. The mixture of BSA and cₑ₆ (lanes 2a and 2b) showed that no fluorescence is retained by the protein band on the gel, thus demonstrating that a fluorescent band localizing with the protein is evidence of covalent conjugation. The lanes of the conjugates (3a and 3b, 4a and 4b) show that a fluorescent band running at the gel front remained after Sephadex chromatography.

The efficiency of the purification by acetone precipitation of the conjugates was confirmed by the gel electrophoresis images shown in FIG. 7C. It can be seen that the fast running fluorescent band disappeared from both the BSA-cₑ₆ and the mal-BSA-cₑ₆ (lanes 2c and 2d, 3c and 3d), while the TLC also showed the disappearance of the fast running spot (FIG. 7A, lanes 2 and 4).

The concentrations of the constituents in the conjugates and, hence the substitution ratios, were measured by absorbance spectroscopy. An aliquot of the conjugate was diluted in approximately 0.1 M NaOH/1% SDS and absorbance between 240 nm and 700 nm scanned. The extinction coefficient of BSA at 280 nm is approximately 47000 cm⁻¹M⁻¹ (Markwell et al. (1978) Anal Biochem 87:206) while the extinction coefficient of cₑ₆ at 400 nm is approximately 150000 cm⁻¹M^{31 1}. Thin layer chromatography was performed on silica gel plates (Polygram SIL G/UV254, Macherey Nagel, Duren, Germany). The chromatograms were developed with an approximately 1:1 mixture of approximately 10% aqueous ammonium chloride and methanol, and spots were observed with fluorescence and absorbance imaging. SDS-PAGE was carried out essentially according to the methods known in the art (Laemmli (1970) Nature 227:680). Gradients of 4-10% acrylamide were used in a non-reducing gel and cₑ₆ was localized on the gel by a fluorometer (excitation at 400-440 nm bandpass filter, emission scanned from 580-720 nm longpass filter (Chemilmager 4000, Alpha Innotech Corp, San Leandro, Calif.). Proteins were localized by Coomassie blue staining.

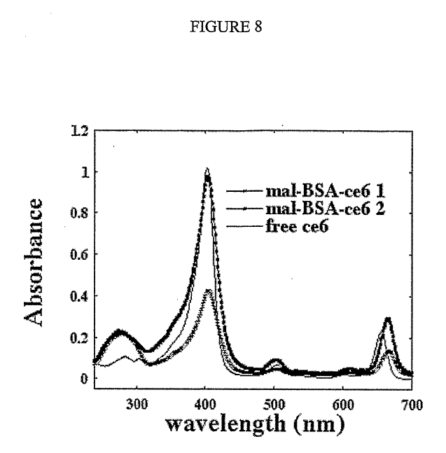

The UV-visible absorption spectra of the purified mal-BSA-cₑ₆ conjugates with the two substitution ratios measured at approximately equal protein concentrations are shown in FIG. 8, together with free cₑ₆ at approximately the same concentration as was present in mal-BSA-cₑ₆ 2. Similar spectra were obtained for BSA-cₑ₆ 1 and 2. Using the values for molar extinction coefficients of BSA at 280 nm of approximately 47000 cm⁻¹M⁻¹ (Markwell et al (1978) Anal Biochem 87:206) and cₑ₆ at 400 nm of approximately 150000 cm⁻¹M⁻¹, and correcting for the small absorbance of cₑ₆ at 280 nm, then the substitution ratios can be calculated to be mal-BSA-cₑ₆ 1 ratio equals approximately 1 protein to approximately 1 dye, and mal-BSA-cₑ₆ 2, ratio equals approximately 1 protein to approximately 3 dye.

### Example 4

### Confocal Fluorescence Microscopy of Rabbit Aorta

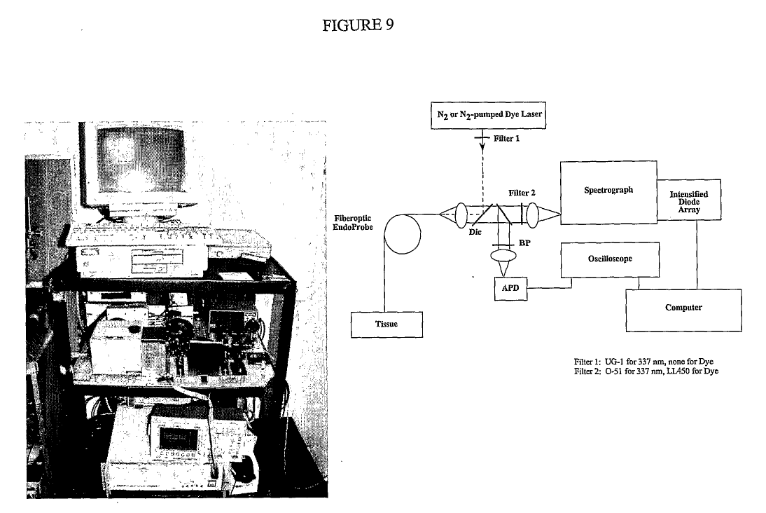

For fluorescence localization within ex vivo aortas, aortic segments were cut open and flattened and the luminal side examined by spectrofluorometry using either a fiber-bundle based double monochromator spectrofluorimeter (Skin Scan, Spex Figure), where emission spectra (excitation 400 nm, emission 580-720 nm) was collected about every 3 mm across the entire area of the exposed intimal surface, or an optical multichannel analyzer (FIG. 9).

For confocal fluorescence microscopy, selected parts of the aortas were snap frozen in liquid nitrogen and approximately 10-20 μm frozen sections were prepared. These sections underwent laser scanning confocal fluorescence microscopy to detect the tissue distribution of the cₑ₆. The red intracellular fluorescence from cₑ₆ together with green tissue auto-fluorescence was imaged in the cells in 10 μm frozen sections. Sections were examined with a laser scanning confocal fluorescence microscope. A Leica DMR confocal laser fluorescence microscope (Leica Mikroskopie und Systeme GmbH, Wetzler, Germany) (excitation 488 nm argon laser) and 4×-40× air immersion lens or a 100× oil immersion objective was used to image at a resolution of 1024×1024 pixels. Two channels collected fluorescence signals in either the green range (580 nm dichroic mirror plus 530 nm (±10 nm) bandpass filter) or the red range (580 nm dichroic mirror plus 590 nm longpass filter) and were displayed as false color images. These channels were overlaid using TCS NT software (Version 1.6.551, Leica Lasertechnik, Heidelberg, Germany) to allow visualization of overlap of red and green fluorescence. These sections were also stained by immunohistochemistry using macrophage specific monoclonal antibodies and conventional H&E staining. Other parts of normal and atherosclerotic aorta were cut into small pieces, weighed and dissolved in sodium hydroxide/SDS and the tissue content of cₑ₆ was determined by spectrofluorimetry as previously described (Hamblin et al (2000) Br. J. Cancer 83:1544).

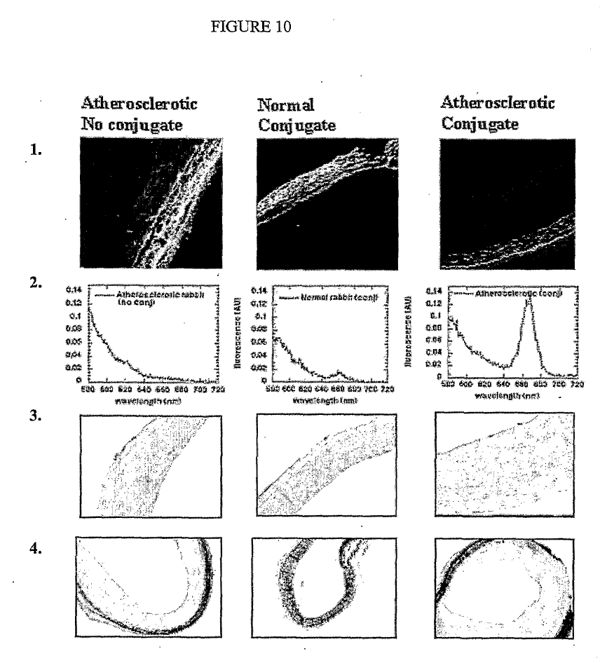

FIG. 10 shows an analysis of aortic sections from rabbits injected with or without conjugate (approximately 2 mg/kg in PBS) about 24 hours after injection of the conjugate. Row 1 shows confocal fluorescence micrographs of frozen aortic sections (red indicates chlorinₑ₆, green indicates elastic lamina auto-fluorescence). Row 2 shows fluorescence emission spectra (excitation at 400 nm) of initmal surface of aortic segments ex vivo. Row 3 shows Hematoxylin and eosin staining of formalin fixed paraffin embedded aortic segments. Row 4 shows Verhoeff s elastic tissue stain. The confocal micrographs showed red fluorescence from the PS (cₑ₆) and green auto-fluorescence principally from the elastic lamina of the arteries. Column 1 shows an atherosclerotic rabbit with no injection of conjugate. There was no red cₑ₆ fluorescence in the tissue section, nor any fluorescence signal from the intimal surface. Column 2 shows a normal non-atherosclerotic rabbit injected with conjugate. There is a small amount of red fluorescence visible in the adventitia rather than the intima in the fluorescence micrographs, and a small fluorescence emission signal from the intimal surface. Column 3 shows an atherosclerotic rabbit injected with conjugate. There was a large amount of red fluorescence visible in the plaque and this gave a corresponding large fluorescence emission signal from the intimal surface.

The intimal fluorescence signal was measured from different sections of aortas from atherosclerotic and normal rabbits. The areas of the abdominal aorta that received balloon injury developed greater amounts of plaque than the neighboring thoracic and lower abdominal aortas. The results from the intimal fluorescence measurements were confirmed by extracting sections of the aortas and measuring fluorescence with a spectrofluorimeter that gives a measure of the number of cₑ₆ molecules in the tissue sections.

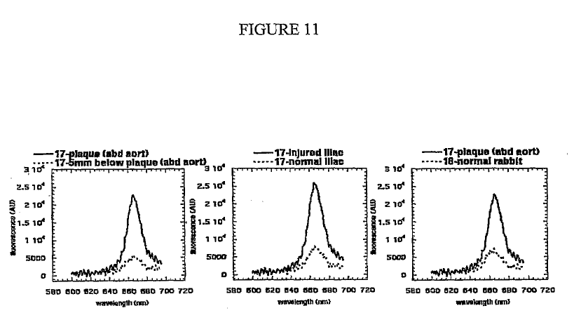

In FIG. 11, a marked contrast was seen between a large aortic plaque and an area of the abdominal aorta 5 mm beneath the plaque (FIG. 11, first panel). In FIG. 11, second panel, another marked contrast was seen between the balloon injured iliac artery and the contralateral normal artery in the same rabbit. Similarly, FIG. 11, third panel, shows a contrast between the plaque-laden aorta of an atherosclerotic rabbit and the same area of the aorta in a normal rabbit.

These data also depict differential imaging between inflamed vs healthy aortic tissue and further support the strategy of employing methods of the invention to treat macrophage-rich vulnerable plaque.

### Example 5

### Enhancement of Inflammatory Cell Uptake of Injected Radiotracers

The ability of an immunomodulator (GMCSF) to enhance detection of active atheromatous and vulnerable plaque was studied in an animal model. Vascular inflammation was induced in three New Zeland white rabbits (with cholesterol feeding and balloon-injury, as described above). Rabbits were imaged 3 hours after injection of FDG (1 mCi/kg) and FDG uptake by their atherosclerotic aorta was determined with Positron Emission Tomography (PET). Thereafter, the rabbits were treated with GM-CSF for 3 days (100 mcg SQ on Days 1 and 2 and IV on Day 3) and FDG imaging was repeated (again, 3 hours after 1 mCi/kg FDG injection). In each case, GMCSF enhanced the ability to detect vascular inflammation (target to background ratio increased by 1.6 to 4.0 fold compared with placebo).

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended numbered claims.

### REFERENCES

Ahmed, N., Kansara, M., and Berridge, M. V.: “Acute regulation of glucose transport in a monocyte-macrophage cell line: Glut-3 affinity for glucose is enhanced during the respiratory burst” 1997. Biochem J. 327 (Pt 2): 369-75.
Akhlynina, T. V., Rosenkranz, A. A., Jans, D. A., and Sobolev, A. S.: “Insulin-mediated intracellular targeting enhances the photodynamic activity of chlorin e6” 1995. Cancer Res. 55(5): 1014-9.
Ausubel, F. M., Brent, R., Kingston, R., Moore, D., Seidman, J., Struhl, K. 1997. Current Protocols in Molecular Biology. John Wiley and Sons, New York, N.Y.
Aveline, B., Hasan, T., and Redmond, R. W.: “Photophysical and photosensitizing properties of benzoporphyrin derivative monoacid ring A (BPD-MA)” 1994. Photochem. Photobiol. 59(3): 328-35.
Babich, J. W., Tompkins, R. G., Graham, W., Barrow, S. A., and Fischman, A. J.: “Localization of radiolabeled chemotactic peptide at focal sites of Escherichia coli infection in rabbits: evidence for a receptor-specific mechanism” 1997. J. Nucl. Med. 38(8): 1316-22.
Bachor, R., Shea, C. R., Gillies, R., and Hasan, T.: “Photosensitized destruction of human bladder carcinoma cells treated with chlorin e6-conjugated microspheres” 1991. Proc. Natl. Acad. Sci. U.S.A. 88(4): 1580-4.
Basu, S., Mukhopadhyay, B., Basu, S. K., and Mukhopadhyay, A.: “Enhanced intracellular delivery of doxorubicin by scavenger receptor-mediated endocytosis for preferential killing of histiocytic lymphoma cells in culture” 1994. FEBS Lett. 342(3): 249-54.
Casscells, W., Hathorn, B., David, M., Krabach, T., Vaughn, W. K., McAllister, H. A., Bearman, G., and Willerson, J. T.: “Thermal detection of cellular infiltrates in living atherosclerotic plaques: possible implications for plaque rupture and thrombosis”. 1996. Lancet 347(9013): 1447-51.
Coligan, J. E., Kruisbeek, A. M., Margulies, D. H., Shevach, E. M., Strober, W. 1991. Current Protocols in Immunology. John Wiley and Sons, New York, N.Y.
Danpure, H. J., and Osman, S.: “Cell labelling and cell damage with indium-111 acetylacetone-an alternative to indium 111 oxine” 1981. Br. J. Radiol. 54(643): 597-601.
Danpure, H. J., Osman, S., and Brady, F.: “The labelling of blood cells in plasma with 111In-tropolonate” 1982. Br. J. Radiol. 55(651): 247-9.
Del Governatore, M., Hamblin, M. R., Piccinini, E. E., Ugolini, G., and Hasan, T.: “Targeted photodestruction of human colon cancer cells using charged 17.1A chlorin e6 immunoconjugates” 2000. Br. J. Cancer 82(1): 56-64.
Eckelman, W. C., and Richards, P.: “Technetium labeling of albumin” 1972. J. Nucl. Med. 13(2): 180-1.
Farb, A., Tang, A. L., Burke, A. P., Sessums, L., Liang, Y., and Virmani, R.: “Sudden coronary death. Frequency of active coronary lesions, inactive coronary lesions, and myocardial infarction”. 1995. Circulation 92(7): 1701-9.
Freeman, M. W.: “Scavenger receptors in atherosclerosis” 1997. Curr. Opin. Hematol. 4(1): 41-7.
Freshney, R. I., 1987. Animal Cell Culture: A Practical Approach. John Wiley and Sons, New York, N.Y.
Gait, M. J. 1984. Oligonucleotide Synthesis: A Practical Approach. IRL Press, Oxford, United Kingdom.
Gijsens, A., and De Witte, P.: “Photocytotoxic action of EGF-PVA-Sn(IV)chlorin e6 and EGF-dextran-Sn(IV)chlorin e6 internalizable conjugates on A431 cells” 1998. Int. J. Oncol. 13(6): 1171-7.
Gunter, K. P., Lukens, J. N., Clanton, J. A., Morris, P. J., Janco, R. L., and English, D.: “Neutrophil labeling with indium-111: tropolone vs. oxine”. 1983. Radiology 149(2): 563-6.
Haberland, M. E., and Fogelman, A. M.: “Scavenger receptor-mediated recognition of maleyl bovine plasma albumin and the demaleylated protein in human monocyte macrophages” 1985. Proc. Natl. Acad. Sci. U.S.A. 82(9): 2693-7.
Hamblin, M. R., and Newman, E. L.: “Photosensitizer targeting in photodynamic therapy. I. Conjugates of haematoporphyrin with albumin and transferrin” 1994. J. Photochem. Photobiol. B. 26(1): 45-56.
Hamblin, M. R., and Newman, E. L.: “Photosensitizer targeting in photodynamic therapy. II. Conjugates of haematoporphyrin with serum lipoproteins” 1994. J. Photochem. Photobiol. B. 26(2): 147-57.
Hamblin, M. R., Rajadhyaksha, M., Momma, T., Soukos, N. S., and Hasan, T.: “In vivo fluorescence imaging of the transport of charged chlorin e6 conjugates in a rat orthotopic prostate tumour” 1999. Br. J. Cancer 81(2): 261-8.
Hamblin, M. R., Del Governatore, M., Rizvi, I., and Hasan, T.: “Biodistribution of charged 17.1A photoimmunoconjugates in a murine model of hepatic metastasis of colorectal cancer”. 2000. Br. J Cancer 83(11): 1544-51.
Hamblin, M. R., Miller, J. L., and Ortel, B.: “Scavenger-receptor targeted photodynamic therapy” 2000. Photochem. Photobiol. 72(4): 533-40.
Hasan, T. (1992) In: B. Henderson and T. Dougherty (eds.), Photodynamic Therapy: Basic Principles and Clinical Applications. Pp. 187-200: Marcel Dekker.
Harlow, E. and Lane, D. 1988. Antibodies: A Laboratory Manual. Cold Spring Harbor Laboratory, New York, N.Y.
Heitner, T., Moor, A., Garrison, J. L., Marks, C., Hasan, T., and Marks, J. D.: “Selection of cell binding and internalizing epidermal growth factor receptor antibodies from a phage display library” 2001. J. Immunol. Methods 248(1-2): 17-30.
Hoffman, E. J., Huang, S. C., Phelps, M. E., Kuhl, D. E. “Quantitation in positron emission computed tomography: 4. Effect of accidental coincidences” 1981. J. Comput. Assist. Tomogr. 5: 391-400.
Hoffman, E. J., Phelps, M. E. “Positron emission tomography: principles and quantitation”. In: Phelps, M., Mazziotta, J., Schelbert, H., eds. Positron emission tomography and autoradiography: principles and applications for the brain and heart. New York: Raven Press; 1986: 237-286.
Jaszczak, R. J. “SPECT: state-of-the-art scanners and reconstruction strategies”. In: Diksic, M., Reba, R. C., eds. Radiopharmaceuticals and brain pathology studied with PET and SPECT. Boca Raton: CRC Press; 1991: 93-118.
Krieger, M.: “Molecular flypaper and atherosclerosis: structure of the macrophage scavenger receptor” 1992. Trends Biochem. Sci. 17(4): 141-6.
Krinick, N. L., Sun, Y., Joyner, D., Spikes, J. D., Straight, R. C., and Kopecek, J. A.: “Polymeric drug delivery system for the simultaneous delivery of drugs activatable by enzymes and/or light” 1994. J. Biomater. Sci. Polym. Ed. 5(4): 303-24.
Laemmli, U. K.: “Cleavage of structural proteins during the assembly of the head of bacteriophage T4” 1970. Nature 227(259): 680-5.
Levy, J. G.: “Photosensitizers in photodynamic therapy”. 1994. Semin. Oncol. 21(6 Suppl 15): 4-10.
Li, A. C., and Glass, C. K.: “The macrophage foam cell as a target for therapeutic intervention”. 2002. Nat. Med. 8(11): 1235-42.
Links, J. M. “Physics and instrumentation of positron emission tomography”. In: Frost, J. J., Wagner, H. N., eds. Quantitative imaging: neuroreceptors, neurotransmitters, and enzymes. New York: Raven Press; 1990: 37-50.
Litvack, F., Grundfest, W. S., Forrester, J. S., Fishbein, M. C., Swan, H. J., Corday, E., Rider, D. M., McDermid, I. S., Pacala, T. J., and Laudenslager, J. B.: “Effects of hematoporphyrin derivative and photodynamic therapy on atherosclerotic rabbits” 1985. Am. J. Cardiol. 1985 56(10): 667-71.
Markwell, M. A., Haas, S. M., Bieber, L. L., Tolbert, N. E.: “A modification of the Lowry procedure to simplify protein determination in membrane and lipoprotein samples” 1978 Anal. Biochem 87(1): 206-10.
McAfee, J. G., and Thakur, M. L.: “Survey of radioactive agents for in vitro labeling of phagocytic leukocytes. I. Soluble agents” 1976. J. Nucl. Med. 17(6): 480-7.
Miller, J. and Calos, M. P. 1987. Gene Transfer Vectors for Mammalian Cells” Cold Spring Harbor Laboratory Press, Cold Spring Harbor, N.Y.
Molpus, K. L., Hamblin, M. R., Rizvi, I., and Hasan, T.: “Intraperitoneal photoimmunotherapy of ovarian carcinoma xenografts in nude mice using charged photoimmunoconjugates” 2000. Gynecol. Oncol. 76(3): 397-404.
Mukhopadhyay, A., Mukhopadhyay, B., Srivastava, R. K., and Basu, S. K.: “Scavenger-receptor-mediated delivery of daunomycin elicits selective toxicity towards neoplastic cells of macrophage lineage”. 1992. Biochem. J. 284(Pt 1): 237-41.
Mullis, K. B., 1994. PCR: The Polymerase Chain Reaction. Birkhauser Press, Cambridge, Mass.
Ochsner, M.: “Photophysical and photobiological processes in the photodynamic therapy of tumours” 1997. J. Photochem. Photobiol. B. 39(1): 1-18.
Parrish, J. A., 1978. Optical properties of the skin and eyes. Plenum Press, New York, N.Y.
Peters, A. M., Saverymuttu, S. H., Reavy, H. J., Danpure, H. J., Osman, S., and Lavender. J. P.: “Imaging of inflammation with indium-111 tropolonate labeled leukocytes” 1983. J. Nucl. Med. 24: 39-44.
Peters, A. M., Danpure, H. J., Osman, S., Hawker, R. J., Henderson, B. L., Hodgson, H. J., Kelly, J. D., Neirinckx. R. D., and Lavender, J. P.: “Clinical experience with 99mTc-hexamethylpropylene-amineoxime for labelling leucocytes and imaging inflammation” 1986. Lancet 2(8513): 946-9.
Phelps, M. E., Hoffman, E. J., Huang, S. C., Ter-Pogossian, M. M. “Effect of positron range on spatial resolution”. 1975. J Nucl Med 16: 649-652.
Polo, L., Segalla, A., Jori, G., Bocchiotti, G., Verna, G., Franceschini, R., Mosca, R., and De Filippi, P. G.: “Liposome-delivered 131I-labelled Zn(II)-phthalocyanine as a radiodiagnostic agent for tumours”. 1996. Cancer Lett. 109(1-2): 57-61.
Plutzky, J.: “Atherosclerotic plaque rupture: emerging insights and opportunities” 1999. Am. J. Cardiol. 84(1A): 15J-20J.
Richter, A. M., Kelly, B., Chow, J., Liu, D. J., Towers, G. H., Dolphin, D., Levy, J. G.: “Preliminary studies on a more effective phototoxic agent than hematoporphyrin” 1987. J. Natl. Cancer Inst. 79(6): 1327-32.
Rockson, S. G., Lorenz, D. P., Cheong, W. F., and Woodburn, K. W.: “Photoangioplasty: An emerging clinical cardiovascular role for photodynamic therapy” 2000. Circulation 102(5): 591-6.
Sacks, F. M., Tonkin, A. M., Shepherd, J., Braunwald, E., Cobbe, S., Hawkins, C. M., Keech, A., Packard, C., Simes, J., Byington, R., and Furberg, C. D.: “Effect of pravastatin on coronary disease events in subgroups defined by coronary risk factors: the Prospective Pravastatin Pooling Project” 2000. Circulation 102(16): 1893-900.
Sambrook, J. 1989. Molecular Cloning: A Laboratory Manual, second edition, Cold Spring Harbor Laboratory Press, New York, N.Y.
Schmidt-Erfurth, U., Diddens, H., Birngruber, R., and Hasan, T.: “Photodynamic targeting of human retinoblastoma cells using covalent low-density lipoprotein conjugates”. Br. J. Cancer 75(1): 54-61.
Sorenson, J. A., and Phelps, M. E. Physics in nuclear medicine, 2nd ed. Philadelphia: W. B. Saunders; 1987.
Sperduto, P. W., DeLaney, T. F., Thomas, G., Smith, P., Dachowski, L. J., Russo, A., Bonner, R., and Glatstein, E.: “Photodynamic therapy for chest wall recurrence in breast cancer” 1991. Int. J. Radiat. Oncol. Biol. Phys. 21(2): 441-6.
Spokojny, A. M., Serur, J. R., Skillman, J., Spears, J. R.: “Uptake of hematoporphyrin derivative by atheromatous plaques: studies in human in vitro and rabbit in vivo” 1986. J. Am. Col. Cardiol. 8(6): 1387-92.
Srivastava, S. C., and Chervu, L. R.: “Radionuclide-labeled red blood cells: current status and future prospects” 1984. Semin. Nucl. Med. 14(2): 68-82.
Svaasand, L. O., and Ellingsen, R.: “Optical properties of human brain” 1983. Photochem. Photobiol. 38(3): 293-9.
Takata, K., Horiuchi, S., and Morino, Y.: “Scavenger receptor-mediated recognition of maleylated albumin and its relation to subsequent endocytic degradation” 1989. Biochim Biophys Acta 984(3): 273-80.
Thakur, M. L., Seifert, C. L., Madsen, M. T., McKenney, S. M., Desai, A. G., Park, C. H.: “Neutrophil labeling: problems and pitfalls” 1984. Semin. Nucl. Med. 14(2): 107-17.
Thakur, M. L., McKenney, S. L., and Park, C. H.: “Evaluation of indium-111-2-mercaptopyridine-N-oxide for labeling leukocytes in plasma: a kit preparation” 1985. J. Nucl. Med. 26: 518-523.
Tsimikas, S., Palinski, W., Halpern, S. E., Yeung, D. W., Curtiss, L. K., and Witztum, J. L.: “Radiolabeled MDA2, an oxidation-specific, monoclonal antibody, identifies native atherosclerotic lesions in vivo” 1999. J. Nucl. Cardiol. 6(1 Pt 1): 41-53.
Vincent, G. M. and Logan, P. M., U.S. Pat. No. 5,422,362
Walther, M. M., Delaney, T. F., Smith, P. D., Friauf, W. S., Thomas, G. F., Shawker, T. H., Vargas, M. P., Choyke, P. L., Linehan, W. M., Abraham, E. H., Okunieff, P. G., and Glatstein, E.: “Phase I trial of photodynamic therapy in the treatment of recurrent superficial transitional cell carcinoma of the bladder” 1997. Urology 50(2): 199-206.
Weir, K. 1996. Handbook of Experimental Immunology Blackwell Science, Inc. Boston, Mass.
Welch, M. J., Thakur, M. L., Coleman, R. E., Patel, M., Siegel, B. A., and Ter-Pogossian, M.: “Gallium-68 labeled red cells and platelets: new agents for positron tomography” 1977. J. Nucl. Med. 18: 558-562.

1. A method of identifying an unstable plaque in a subject, said method comprising the steps of:
a) administering a diagnostic agent;
b) administering an immune modulator that increases localization of the diagnostic agent to inflammatory cells of the plaque;
c) detecting a sufficient amount of the diagnostic agent to thereby identify the plaque in the subject.

2. The method of claim 1, wherein the unstable plaque is an active atheromatous plaque.

3. The method of claim 1, wherein the unstable plaque is a vulnerable plaque.

4. The method of claim 3, wherein the vulnerable plaque comprises inflammatory components, a large lipid pool, and a thin fibrous cap.

5. The method of claim 4, wherein inflammatory components are selected from the group consisting of inflammatory cells, lipids, procoagulants and agents that promote inhibition of extracellular matrix production or degradation of extracellular matrix.

6. The method of claim 3, wherein the thin fibrous cap of the vulnerable plaque is less than about 150 microns thick.

7. The method of claim 3, wherein the thin fibrous cap of the vulnerable plaque is less than about 100 microns thick.

8. The method of claim 1, wherein the inflammatory cells are selected from the group consisting of smooth muscle cells, dendritic cells, follicular dendritic cells, Langerhans cells, interstitial, interdigitating, blood, and veiled dendritic cells, leukocytes, natural killer cells, lymphocytes, monocytes, macrophages, alveolar macrophages, microglia, mesangial cells, histiocytes, Kupffer cells, foam cells, mast cells, endothelial cells, megakaryocytes, platelets, erythrocytes and polymorphonuclear cells.

9. The method of claim 8, wherein the lymphocytes are B-lymphocytes or T-lymphocytes.

10. The method of claim 8, wherein the polymorphonuclear cells are granulocytes, basophils, eosinophils or neutrophils.

11. The method of claim 5, wherein the inflammatory cells consist of greater than about 10% macrophages and/or monocytes.

12. The method of claim 5, wherein the inflammatory cells consist of greater than about 25% macrophages and/or monocytes.

13. The method of claim 4, wherein the lipid content of the plaque is greater than about 10%.

14. The method of claim 4, wherein the lipid content of the plaque is greater than about 25%.

15. The method of claim 1, wherein the immune modulator is selected from the group consisting of colony stimulating factors, interleukins, interferons, chemokines, chemoattractants, growth factors, inhibitory factors, bacterially derived epitopes and signal transduction molecules.

16. The method of claim 1, wherein the immune modulator is selected from the group consisting of GM-CSF, M-CSF, G-CSF, interleukin-1 to -29, TNFα, formyl-methionine-leucine-phenylalanine (fMLP), lipopolysaccharide (LPS), phorbol 12-myristate-13-acetate, interferon α, interferon β, interferon γ, CD40, ligands of CD40, gp39, monocyte chemoattractant protein, basic fibroblast growth factor (bFGF), muramyl dipeptide, urokinase, regulated upon activation normally T-cell expressed and presumably secreted (RANTES), growth regulated oncogene, interferon-inducible T-cell alpha chemoattractant (I-TAC), monokine induced by gamma-interferon (MIG-1), leukemia inhibitory factor (LIF), oncostatin M, transforming growth factor β (TGF β), tissue inhibitor of matrix metalloproteinases (TIMP), macrophage chemotactic factor (MCF), and macrophage inflammatory protein.

17. The method of claim 1, wherein the diagnostic agent is selected from the group consisting of a photosensitizer, fluorescent marker and radiolabeled marker.

18. The method of claim 17, wherein the photosensitizer is motexafin lutetium.

19. The method of claim 17, wherein the photosensitizer is chlorinₑ₆.

20. The method of claim 17, wherein the photosensitizer is MV0633.

21. The method of claim 17, wherein the fluorescent marker is Fluorodeoxyglucose.

22. The method of claim 17, wherein the radiolabeled marker is β-emitter.

23. The method of claim 22, wherein the β-emitter is selected from the group consisting of ¹³¹I, ¹²⁵I, ¹²³I, ^{99m}Tc, ¹⁸F, ⁶⁸Ga, ⁶⁷Ga, ⁷²As, ⁸⁹Zr, ⁶²Cu, ¹¹¹Cu, ²⁰³In, ¹⁹⁸Pb, ¹⁹⁸Hg, ⁹⁷Ru, ¹¹C, Re¹⁸⁸ and ²⁰¹Tl.

24. The method according to claim 17, wherein the β-emitter is ¹⁸F-Fluorodeoxyglucose.

25. The method according to claim 17, wherein the β-emitter is ¹⁸⁸Re.

26. The method according to claim 17, wherein the diagnostic agent is a radiolabeled marker and the signal emitted by the radiolabeled marker is detected by positron emission tomography, magnetic resonance imaging, computer tomography, single photon emission computed tomography or a β-ray detector probe.

27. The method of claim 1, wherein the diagnostic agent is coupled to a molecular carrier.

28. The method of claim 27, wherein the molecular carrier targets the diagnostic agent to inflammatory cells selected from the group consisting of smooth muscle cells, dendritic cells, follicular dendritic cells, Langerhans cells, interstitial, interdigitating, blood, and veiled dendritic cells, leukocytes, natural killer cells, lymphocytes, monocytes, macrophages, alveolar macrophages, microglia, mesangial cells, histiocytes, Kupffer cells, foam cells, mast cells, endothelial cells, megakaryocytes, platelets, erythrocytes and polymorphonuclear cells.

29. The method of claim 28, wherein the lymphocytes are B-lymphocytes or T-lymphocytes.

30. The method of claim 28, wherein the polymorphonuclear cells are granulocytes, basophils, eosinophils or neutrophils.

31. The method of claim 27, wherein the molecular carrier is selected from the group consisting of serum proteins, receptor ligands, microspheres, liposomes, antibodies, growth factors, peptides, hormones and lipoproteins.

32. The method of claim 27, wherein the molecular carrier binds to a scavenger receptor.

33. The method of claim 32, wherein the molecular carrier is selected from the group consisting of maleylated albumin, daunorubicin, doxorubicin, oxidized low density lipoprotein, acetylated low density lipoprotein, oxidized high density lipoprotein, malondialdehyde treated proteins, formaldehyde treated albumin, glycated albumin, polyinosinic acid, glycated lipoproteins, dextran sulfate, anionic phospholipids, fucoidin, carrageenan, polyvinyl sulfate and monoclonal antibodies that recognize CD11b, CD11c, CD13, CD14, CD16a, CD32 or CD68.

34. The method of claim 33, wherein the anionic phospholipid is phosphatidyl serine.

35. The method of claim 27, where in the molecular carrier targets the diagnostic agent to a T-cell.

36. The method of claim 35, wherein the molecular carrier targets the diagnostic agent to a T cell biomolecule selected from the group consisting of IL-10, IL-10 receptor, monocyte inflammatory protein-1, monocyte inflammatory protein-1 receptor and transferrin.

37. The method of claim 35, where in the molecular carrier is selected from the group consisting of monoclonal antibodies that recognize CD1, CD2, CD3, CD4, CD5, CD6, CD7, CD8, CD25, CD28, CD44 and CD71 and transferrin.

38. The method of claim 27, where in the molecular carrier targets the diagnostic agent to lipids of the plaque.

39. The method of claim 38, wherein the molecular carrier comprises hydrophobic vehicles selected from the group consisting of liposomes, cremaphor EL, PEG/solvent mixtures, iodized castor oil, nanoparticles and micellar preparations.

40. The method of claim 39, wherein the liposomes contain cholesterol.

41. The method of claim 39, wherein the liposomes contain cardiolipin.

42. The method of claim 27, wherein the molecular carrier targets the diagnostic agent to macrophages.

43. The method of claim 42, wherein the molecular carrier targets the diagnostic agent to a macrophage biomolecule selected from the group consisting of For-Met-Leu-Phe, tenascin C, tissue factor, tissue inhibitor of MMP 1, tissue inhibitor of MMP 2, oxidized LDL receptor, heme oxygenase-1, human cartilage gp-39, IL-6, IL-6 receptor, IL-10, IL-10 receptor, lectin-like oxidized LDL-receptor, monocyte inflammatory protein-1, monocyte inflammatory protein-1 receptor and macrophage chemoattractant protein-1 receptor.

44. The method of claim 27, wherein the molecular carrier targets the diagnostic agent to foam cells.

45. The method of claim 27, wherein the molecular carrier that targets the diagnostic agent is a protease that degrades extracellular matrix.

46. The method of claim 45, wherein the protease is a metalloproteinase.

47. The method of claim 45, wherein the molecular carrier is a monoclonal antibody that binds to an epitope on a protease.

48. The method of claim 1, wherein the subject is a human.

49. A method of identifying an unstable plaque in a blood vessel of a subject, said method comprising the steps of:
a) administering a diagnostic agent;
b) administering an immune modulator that increases localization of the diagnostic composition to inflammatory cells of the plaque;
c) comparing a signal emitted by the diagnostic agent in one area of the blood vessel to a signal emitted by the diagnostic composition in another area of the blood vessel; and
d) determining the location of the greater amount of signal to thereby identify the plaque in the subject.

50-96. (canceled)

97. A method of treating an unstable plaque in a subject, said method comprising the steps of:
a) administering a therapeutic agent;
b) administering an immune modulator that increases localization of the therapeutic agent to inflammatory cells of the plaque; and
c) stabilizing, reducing or eliminating the plaque, thereby treating the subject for the unstable plaque.

98-113. (canceled)

114. A method of identifying and stabilizing an unstable plaque in a subject, said method comprising the steps of:
a) administering at least one photosensitizer;
b) administering an immune modulator that increases localization of the photosensitizer to inflammatory cells of the plaque;
c) detecting a sufficient amount of the photosensitizer to thereby identifying the plaque; and
d) irradiating the photosensitizer to produce a phototoxic species that stabilizes the plaque in the subject.

115-130. (canceled)

131. A kit for detecting an unstable plaque comprising a diagnostic agent, an immune modulator and instructions for using the diagnostic agent and the immune modulator to identify unstable plaque in accordance with the method of claim 1.

132-133. (canceled)

134. A kit for treating an unstable plaque in a subject in need thereof comprising a therapeutic agent, an immune modulator and instructions for treating the unstable plaque using the therapeutic agent and the immune modulator in the subject in accordance with the method of claim 97.

135. (canceled)

136. A kit for detecting and treating an unstable plaque in a subject in need thereof comprising a photosensitizer, an immune modulator, and instructions for using the photosensitizer and the immune modulator to detect and treat the unstable plaque in the subject in accordance with the method of claim 114.
